(19) United States
(12) Patent Application Publication
(10) Pub. No.: US 2026/0045367 A1
(43) Pub. Date: Feb. 12, 2026

(54) AUTOMATED ANOMALY DETECTION AND RESPONSE GENERATION IN A SENSOR-ENABLED ENVIRONMENT

(71) Applicant: LogicMark, Inc., Louisville, KY (US)

(72) Inventors: Chia-Lin Simmons, Moraga, CA (US); Rafael Saavedra, Cupertino, CA (US); Peter Williams, Belmont, CA (US)

(21) Appl. No.: 19/296,073

(22) Filed: Aug. 11, 2025

Related U.S. Application Data
(60) Provisional application No. 63/682,045, filed on Aug. 12, 2024.

(51) Int. Cl. G16H 50/30; G05B 9/02; G16H 10/60

(52) U.S. Cl. CPC G16H 50/30; G05B 9/02; G16H 10/60

(57) ABSTRACT

Automated anomaly detection and response generation in a sensor-enabled environment (SEE) may be provided via measuring, via a plurality of sensors disposed in a SEE, state information of the SEE and a person under monitoring (PUM) within the SEE; identifying, using the state information, an in-progress behavior affecting the PUM in the SEE; identifying an intended outcome of the in-progress behavior; predicting, using the state information and the in-progress behavior, a predicted outcome of the in-progress behavior; and in response to identifying that a difference between the intended outcome and the predicted outcome represents an actionable risk level for health, wellness or safety of the PUM: identifying a responsive action to the in-progress behavior to reduce the actionable risk level to the PUM; and performing the responsive action.

### CROSS-REFERENCES TO RELATED DISCLOSURES

The present disclosure claims priority to U.S. Provisional Patent Application 63/682,045, filed on 2024 Aug. 12, titled “AUTOMATED ANOMALY DETECTION AND RESPONSE GENERATION IN A SENSOR-ENABLED ENVIRONMENT”, which is incorporated herein in its entirety.

### BACKGROUND

A sensor-enabled environment (SEE) is a space in which one or more electronic sensors interact with an observing system such that the observing system can ascertain a state of one or more persons under monitoring (PUM) domiciled within the SEE. A PUM may, for example, be an elderly individual who requires observation due to a health condition and a corresponding SEE may, for example, be the home of that individual. Sensors of the SEE can include worn fitness trackers, motion sensors, thermometers/thermostats, cameras, light and radio range finding devices, microphones, and weight-sensitive devices. The observing system may communicate with one or more individuals outside the SEE to provide updates regarding a status of the PUM.

### SUMMARY

Risk identification and management in a sensor-enabled environment is provided herein. An example method comprises receiving data about a person under monitoring (PUM) from a sensor enabled environment (SEE), calculating at least one risk metric for the PUM based on the data from the SEE, determining, based on the at least one risk metric, that at least one safety metric correlated to the at least one risk metric will likely be or already is outside of a predefined safe range, and alerting a care provider of the PUM that a safety event is occurring or imminent.

### BRIEF DESCRIPTION OF FIGURES

The present disclosure may be understood in reference to the following Figures. It will be noted that these Figures are presented for illustrative and exemplary purposes only and are not to be construed as limiting in any way. It will also be appreciated that features of two or more of the following Figures may be freely combined or omitted as desired.

FIG. 1 illustrates an example system for assessing risk metrics in a sensor-enabled environment, according to example embodiments of the present disclosure.

FIG. 2 illustrates an example of data flow in risk and safety systems, according to example embodiments of the present disclosure.

FIG. 3 illustrates an example of predictive and risk measurement systems employing digital twins, according to example embodiments of the present disclosure.

FIG. 4 illustrates an example of data flow in safety evaluation systems, according to example embodiments of the present disclosure.

FIG. 5 illustrates an example of risk, predictive, and safety systems' interactions, according to example embodiments of the present disclosure.

FIG. 6 is a flowchart of an example method for automated anomaly detection and response generation in a sensor-enabled environment, according to embodiments of the present disclosure.

FIG. 7 is a flowchart of an example method for automated anomaly detection and response generation in a sensor-enabled environment, according to embodiments of the present disclosure.

FIG. 8 illustrates an example computing device, as may be used as a controller in a SEE to monitor a PUM, as part of a sensor monitoring a PUM, as part of a central or distributed service providing calibration systems for generating and curating AI/ML models for distribution to the SEEs, and the like, according to embodiments of the present disclosure

### DETAILED DESCRIPTION

The use of monitoring to support a person under monitoring (PUM) in a sensor enabled environment (SEE) can include the provision of one or more risk assessment or estimation systems coupled with one or more response systems. In some embodiments, these capabilities may be integrated to provide a monitored environment that can, in whole or in part, evaluate the health, wellness or safety risk(s) for one or more PUM through the behaviors, habits or activities of those PUMs within that environment. This risk evaluation data can inform one or more response systems, which may then communicate these risk evaluations to one or more other systems or stakeholders, including for example, emergency response teams, to avoid or mitigate any detected risks. Inherent in any response system is the potential or actual impact of those responses for or on the PUM, and as such, in some embodiments, one or more impact assessment systems may operate in collaboration, for example, as part of a dynamic feedback mechanism to further consider, evaluate, model or predict these impacts, which can in turn lead to those responses being modified or varied so as to effect the most beneficial outcome for a PUM in the prevailing circumstances.

A PUM may be any person who has one or more sensors, devices or systems that are, at least in part, employed to monitor that person to evaluate the wellness, health or safety of that person as that person undertakes the behaviors that unfold as part of living life.

A risk metric may be a measured scalar or vector indicative of a degree or type of risk to which a behavior, activity or event exposes a PUM. For example, a safety metric may be derived, mathematically or otherwise, from one or more risk metrics and may be a scalar or vector, which can be presented to a stakeholder, for example in human-readable form, so that the stakeholder can assess a risk status of the PUM. Calculation of risk metrics may also take perceptual indications of the PUM, such as an indication that the PUM feels or declares that they are at risk, for example, unsafe, into account.

One aspect of this approach may be the recognition and understanding of the context for any one or more responses and the impacts thereof. For example, context of the risks and consequent responses can include, the health, wellness or safety of the PUM, which can include the Heath Care Profile (HCP) of the PUM, which can be the specification of a current health condition of the PUM that is, at least in part, the reason for monitoring the PUM. The HCP can include, for example, data sets generated by the one or more sensors, devices or systems comprising the Sensor Enabled Environment (SEE), which in some embodiments can include one or more Personal Physics Engines (PPE). In some cases, the use of a SEE for a PUM may be preventative, in that, for example, the age of a person may be determinate of monitoring that person in a SEE.

The intention of monitoring, for example as determined by the HCP of the PUM or as a preventive monitoring for the safety or wellbeing of the PUM can encompass a number of aspects of the behaviors, activities, and undertakings of the PUM, including for example, physical mobility, mental acuity, financial safety and stability, relationships, social interactions, one or more health conditions or the overall wellness and wellbeing of the PUM.

In some embodiments, one or more care response systems may be invoked to undertake, for example, identification, categorization, evaluation, analysis, prediction, verification or validation of the one or more risks that can be pertinent to one or more PUM in a SEE. These systems can include, for example, inputs, risk evaluation systems, response systems, impact analysis systems, action execution systems, digital twins, Artificial Intelligence (AI)/Machine Learning (ML) systems (including large language models (LLM), Large concept Models (LCM), specialist models and the like), Personal Physics Engines (PPE), game theory systems, and combinations thereof.

One further aspect of some embodiments can be the use of feedback or feedforward in the processes of risk measurement, evaluation, prediction, response, impact or execution systems. This usage may be in part to avoid undertaking an action that can in certain circumstances have unintended consequences where, for example, that action may have, in whole or in part, a detrimental impact. The use of feedback and feedforward techniques as described herein can be used to mitigate potential or actual responses or actions, that seen in isolation, may appear to be beneficial, yet with a more comprehensive understanding of the context through employing these techniques, can ensure that these responses or actions provide the intended benefits, while reducing (e.g., minimizing) or avoiding any unintended consequences.

The safety, health or wellness of a PUM in a set of circumstances can be, at least in part, determined by the behaviors of the PUM, in that the behaviors can be similar to the routine behaviors of the PUM and these behaviors, may not involve any actions that have the potential to impact the safety, health or wellness of the PUM in such circumstances, from the perspective of evaluating risk or potential responses.

This state of safety, health or wellness can, in some embodiments, be calculated from the one or more patterns forming such behaviors that are based, in part of in whole, on the data sets generated by the one or more sensors, devices or systems present in a SEE.

In some embodiments, a PUM may self-declare their safety to one or more SEE systems, for example, a care hub or care processing system. Such declarations can include, for example, declarations of the PUM feeling unsafe or having other concerns as to their wellbeing, which can, in some embodiments, be received by the one or more sensors, devices or systems present in a SEE, for example a Personal Emergency Response System (PERS) device, smart phone, Micro Electrical Mechanical (MEMs) microphone or other acoustic capture devices, or other biometric devices or the like.

In some embodiments, one or more sensors, devices or systems may comprise a SEE, where at least one PUM is domiciled. The SEE may generate data sets that are representations of the behaviors of the one or more PUM in the context of the SEE, where those data sets include inputs to, for example, one or more risk or response systems.

Further inputs to such systems may be generated by, for example, the PUM, in the form of actions, behaviors, commands, communications or specifications, for example as expressed in the HCP of the PUM.

In some embodiments, there may be one or more systems that are employed for representation of the behaviors or activities of the PUM in, for example, a digital twin of the PUM and the environment of the PUM. This representation can include, for example, one or more Personal Physics Engine (PPE), care hubs or care processing systems which can generate one or more data sets, for example as tokens, patterns, frameworks or other organizations, which can be communicated as inputs to one or more risk or response systems, such as for example a Care Risk Response Management system (CRRM).

In some embodiments, risk metrics can be used, in whole or in part to generate one or more communications, including to one or more sensors, devices or systems or to one or more stakeholders, for example, a carer, friend, family member or the PUM, to advise that there is an actual or predicted increase in one or more risk or safety metrics, which can indicate a safety, health or wellness issue. These communications may provide sufficient notice of such issues, so that the stakeholders, including the PUM, may avoid or mitigate the risk. For example, if a floor is wet, this issue may be communicated to a PUM and potentially to a carer, so that the PUM may then not traverse the wet surface, or in which the carer may assist the PUM in that traversal to thereby avoid or mitigate the risk of falling.

A similar approach can be employed where, although the risk has increased, an event or activity still may occur; however, the communications may enable the risk and any event or activity causing any safety, health or wellness impact to be mitigated. For example, this mitigation can include, where possible, delaying an action or event until the risk or safety levels reach an acceptable level, by communicating with one or more stakeholders to render assistance to, for example, a PUM, as the PUM encounters or undertakes the event, action, activity or the like. One consideration of such mitigation can be the reduction in consequences of the activity, event, action or other occurrence, with can include the identification of, for example, unintended consequences. For example, if a PUM takes a particular medication and then undertakes a particular activity, there may be an untended consequence of an increased risk of falling, through for example dizziness.

In some embodiments, risk or other safety metrics can be employed, in whole or in part, to optimize, including by selection, priority, time, location or other characteristics, one or more responses to an event, activity or other occurrence which the PUM is or will experience. This optimization can include, for example, calculation of the one or more responses and the potential impacts of the responses on the PUM or other stakeholders, such that the response is optimized for the safety, health or wellbeing of the PUM. For example, this optimization can include determining for a set of possible responses, for example, calling one or more stakeholders, including a carer, friend or family member or configuring one or more sensors, devices or systems to generate more specific and detailed data sets for the event, activity or occurrence or communicating with the PUM, for example, in an interactive manner, for example, including the use of one or more AI/ML systems configured for such interactions, where, for example, the PUM has tripped and suffered a toe injury, where the optimized response is supporting the PUM's wellbeing without making unnecessary, and potentially expensive, responses, such as calling out to emergency services (e.g., ambulances, paramedic, fire department, or other first responders).

An aspect of this approach is the consideration or evaluation of the outcomes of such responses, through evaluation of the outcomes and the actual or potential impact on the safety, health or wellbeing of the PUM or other stakeholders. For example, if the PUM experiences dizziness on a consistent basis, for example, when rising from sitting, this action may have an elevated risk or safety metric for that activity, which can be communicated to one or more other stakeholders, including, for example, medical professionals, carers and the like, such that, for example, the medications of the PUM can be varied to decrease such experiences.

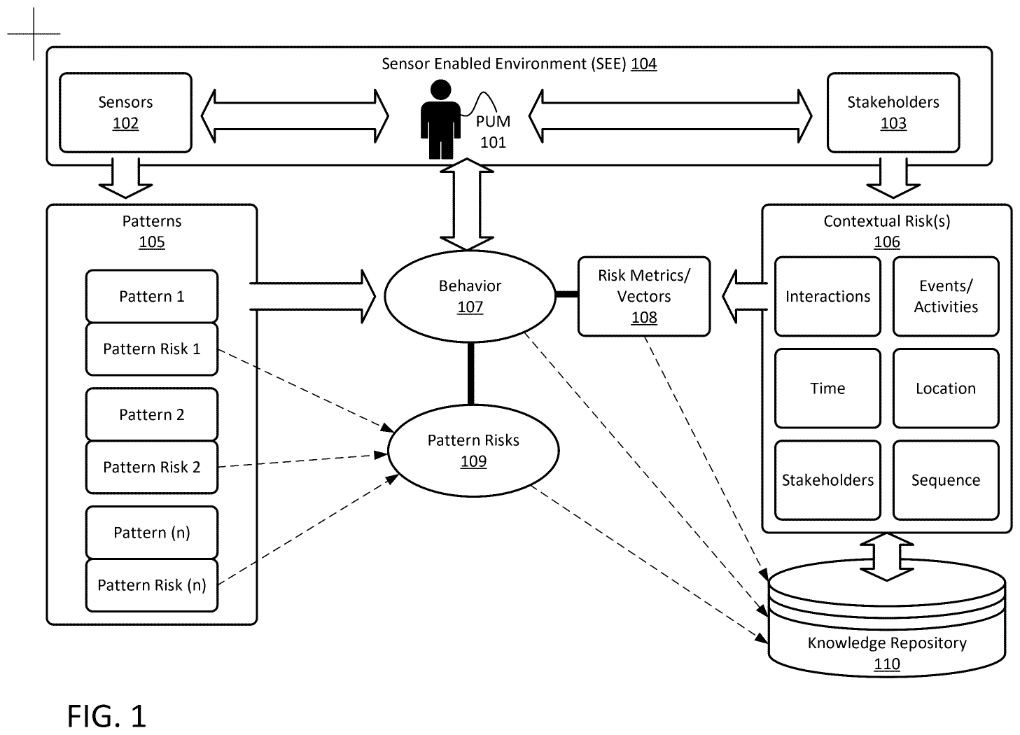

FIG. 1 illustrates an example embodiment, where a PUM 101 or one or more stakeholders 103 are present in a SEE 104 that includes one or more sensors, devices or systems 102. These sensors, devices or systems generate one or more data sets that contain one or more patterns 105, where for example pattern 1, 2 . . . (n) are represented. As used herein, a pattern may be one or more behaviors of the PUM that may commonly occur or that may deviate from a normal behavior in a more or less consistent manner or degree. Each of these patterns can have one or more risk metrics, described as the pattern risks, measuring the one or more risks of that pattern. These patterns can, in some embodiments, for one or more behaviors 107, which represent the PUM 101 activities. These behaviors 107 can have one or more sets of pattern risks 109, which in some embodiments are the aggregate of the pattern risk of each individual pattern. Such aggregation can, in some embodiments, involve one or more functions, such as additive, derivative, integral, or other combinatorial expression. These behaviors 107 can also have one or more contextual risks, represented for example, by one or more risk metrics or vectors 108. These risk metrics or vectors 108 can include one or more contextual risks 106, which can include time, location, sequences, interactions, Events and activities or stakeholders in any arrangement, where each of these elements can have one or more risk metrics. The combinations of these contextual risks 106 can include one or more features or variables, such that certain dependencies and other relationships may be represented. In some embodiments, such contextual risks 106, risk metrics and vectors 108, behaviors 107, pattern risks 109 and other pertinent data, including patterns 105 can be stored in one or more knowledge repository.

In some embodiments, one or more sensors, devices, or systems may form part of a SEE, where at least one PUM is primarily domiciled. The SEE may generate data sets that are measurements or representations of the behaviors of the one or more PUM in the context of the SEE.

Such representations may comprise sets of data, for example, in the form of detected patterns, frameworks, tokens or other organizations or arrangements. Each of these representations may have various attributes and characteristics, which can be used by one or more systems, including those of the SEE including care hubs or care processing systems.

In some embodiments, there may be one or more risk attributes that are bound, by reference or embedding to these representations. For example, a framework, such as a mobility framework representing a person moving from sitting to standing, may have one or more risk attributes, expressed for example, as risk metrics, bound to that activity.

In some embodiments, these risk attributes may have one or more dependencies, based at least in part, on, for example the time of day, location, PUM behavior (of which such mobility framework is part thereof), context, for example a knock at the door or rain outside or other contextual or environmental considerations. In some embodiments, such considerations may be identified by the one or more sensors, devices, or systems comprising the SEE, including for example care hubs or care processing systems.

Such risk attributes may be in the form of a set of risk metrics, which are described herein. Each of the risk attributes may comprise one or more risk metrics, which may be in the form of a set, linked list, sequence, taxonomy, ontology, lattice, or other organizational arrangements.

One aspect of this approach may be the representation of risk as one or more attributes, where such risk is represented in a “bottom up” method, in that each of the segmented, quantized or other representations of the behaviors of the PUM has one or more bound risk attribute. This representation can include the use of tokenization, where for example a segment or section of data is tokenized, for example in the form of a single token or a multi-segment token and each of the tokens of sections thereof can have one or more risk metric bound, through reference or embedding.

In some embodiments, one or more response systems may operate, based at least in part, on data sets generated by the one or more sensors, devices or systems present in a SEE, where such data sets are processed by one or more systems, including, for example, care hubs, care processing systems or risk evaluation systems in any arrangement, where such systems undertake one or more evaluations of such data sets, including using one or more AI/ML systems, and generate one or more communications that can be communicated to one or more response systems.

In this example, the configuration of a response system may include processes such as communications including notifications, or generation of one or more outcomes, for example in the form of further communications, such as calibration, configuration or other specifications for the one or more sensors, device or systems comprising the SEE. Such communications can include those communicated, for example, directly to a PUM or other stakeholder through for example, their embedded, carried or worn sensors, devices or systems, using for example, haptic, audio, video or text in any arrangement.

In some embodiments, response systems may incorporate one or more agents, which can represent such systems and can be embedded in or accessed by one or more sensors, devices or systems, including those being carried, worn or embedded in or on a PUM or other stakeholder. For example, a response system may include a set of agents that interact with one or more control functions to provide such communications to the one or more PUM or stakeholders.

In some embodiments, for example, response systems may communicate with safety enabled devices, described herein, to provide a PUM or other stakeholder with messages, configurations or other communications regarding their current or future safety. For example, this can include communications that include warnings as to the safety of a current or future activity of a PUM or other stakeholder.

In some embodiments, a response system may invoke one or more Digital Twin, AI/ML system, Game theory module, or personal physics engine (PPE) to predict, in whole or in part, behaviors of a PUM or other stakeholders that can result in one or more events that can be detrimental to the safety, health or well-being of the PUM. These responses can include the prediction of one or more patterns or behaviors which can have a negative, positive or neutral health, wellness or safety impact.

In some embodiments, response systems may invoke one or more impact analysis systems where, for example, the one or more outcomes of one or more response systems can be evaluated for the potential impact of that response. For example, a response that addresses one aspect of the PUM situation, for example, blood pressure issues causing dizziness, may have a further impact on, for example the mobility of the PUM.

One further aspect of the response systems is the degree of immediacy of any action and consequent potential or actual impact of that action on PUM or other stakeholders. The degree of immediacy can include one or more system global or local feedback/feedforward loops so that the potential responses can be integrated into the calculation of the risks for those impacts. These potential responses can include those predicted by the one or more prediction systems, including AI/ML systems, game theory modules, digital twins or other care processing systems in any arrangement.

In some embodiments, response systems may generate candidate responses, where, for example, there are a number of potentially beneficial response candidates. These candidates can have one or more selection criteria, for example, those selected by the PUM, other stakeholders or by one or more systems, such as for example a care hub or care processing system.

One such example criterion could be the potential impact of each of the candidate responses, for example, there may be known allergies to or side effects of a particular food, beverage, medicine, activity or other response. These potential responses may, in some embodiments, be evaluated using, for example, one or more PPE, digital twin or AI/ML system.

In some embodiments, the outputs of one or more response systems may be evaluated by one or more impact analysis systems (IAS). The IAS can, in some embodiments, form part of a feedback system intended to evaluate one or more response systems outcomes using, for example, one or more digital twins, PPE, game theory engines or AI/ML, including LLM or LCM systems in any arrangement.

The evaluation of such responses can include invocation of one or more digital twins of the environment or the PUM, including for example a PPE, to represent the current or future situation of the PUM in an environment. The communications from the response systems, or other risk evaluation systems, may then be instantiated in the digital twins, so as to determine the likely outcomes, using for example, one or more AI/ML systems or other predictive systems, including for example, an LCM or a Large Language Model (LLM) with retrieval augmented generation (RAG), which can include a PPE. This inclusion can include using the known impact criteria, for example side effects of medications, impact of strenuous activities of the PUM, known effects of foods or beverages and any other previously observed or measured impacts on the PUM. The use of impact criteria can, for example, include impacts or effects that have been declared by the PUM or other stakeholders, such as a caretaker, family member, medical practitioner and the like.

The provision of impact criteria from a stakeholder can include, for example, where the response systems have generated a communication to, for example, the PUM or other stakeholders or sensors, devices or systems comprising the SEE, and the IAS may evaluate those communications for potential impacts, including misalignments of any incentives for the PUM or other stakeholders.

One aspect of the IAS can be the ability, in some embodiments, to use incentive analytics, where, for example, the incentives of one or more stakeholders, including the PUM can be evaluated to identify any incentive misalignments, which can be represented in the form of incentive metrics. For example, the identification can include positive, negative and neutral metrics that indicate the propensity of the incentives, as expressed directly or indirectly, by the stakeholder to align with the perceived incentives of that stakeholder. Such analytics may be used, in part or in whole, to represent further risk or safety metrics.

In some embodiments, the use of one or more digital twins, that, for example, include or interoperate with one or more AI/ML systems can generate AI/ML assisted or risk reduction or mitigation strategies that can be deployed. These strategies may then be communicated to one or more action execution systems (AES).

One aspect of the IAS may be the evaluation of actions versus non-action, where, for example, such systems may generate a further set of metrics, for example, expressed as weightings, for one or more responses or outcomes under consideration. This evaluation can include, for example, representing the evaluated responses or outcomes in the form of a set of new objectives, which can be used as a feedback mechanism.

In some embodiments, the IAS can employ one or more decision mechanisms, including decision matrices, game theory games, AI/ML systems and other predictive embodiments.

In some embodiments, one or more AES may be invoked to undertake one or more activities or actions, such as communicating with one or more sensors, devices or systems comprising the SEE or communicating with one or more PUM or other stakeholders, including stakeholders that may be remote from the SEE, such as healthcare professionals, emergency services, delivery services, food and other provision providers and the like. The undertaking of an action or activity can include the use of one or more predictive systems such as for example, digital twins, AI/ML modules or game theory modules to anticipate potential invocations, including, for example the potential impact of those invocations, using for example an IAS.

In some embodiments, an AES can deliver specifications including commands, calibration data, configuration data, or other data to those systems capable of providing an execution capability compatible with those data. These specification can be communicated to systems that have control interfaces, such as for example, Heating Ventilation and Air Conditioning (HVAC) systems, home automation systems, IoT devices, services such as transport, food or other delivery, health or other care providers or the like.

In some embodiments, the delivering of specifications can include providing corresponding communications to the one or more PUM or other stakeholders regarding the invocation of these one or more systems.

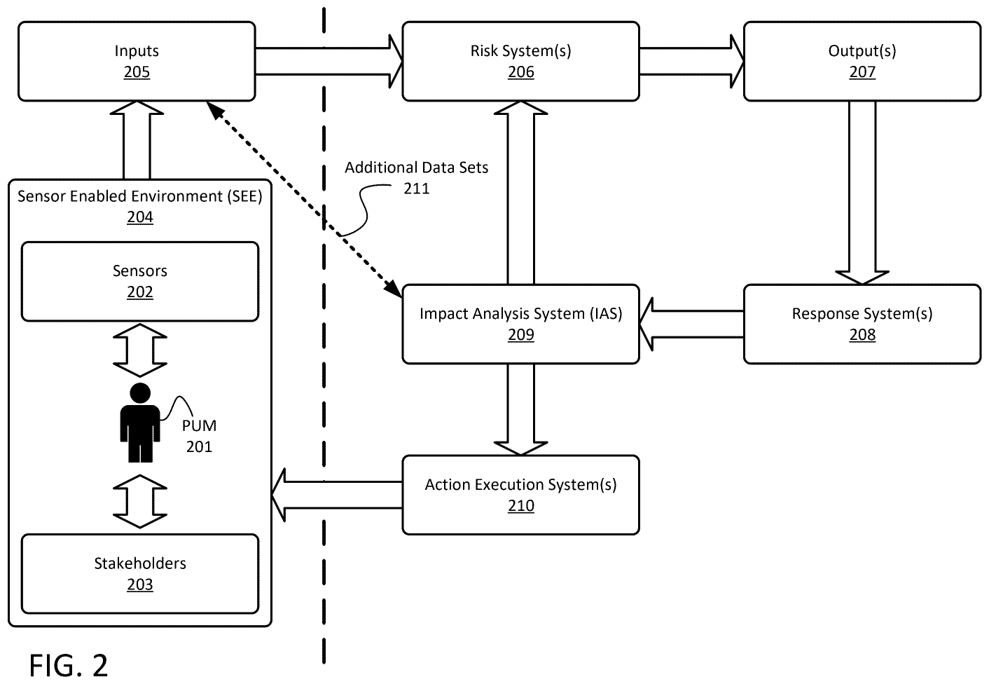

FIG. 2 illustrates an example embodiment where a PUM 201 is present in a SEE 204, which includes one or more sensors, devices or systems 202 or one or more stakeholders 203. The SEE can generate one or more data sets including those from the one or more sensors, devices or systems and those from the activities of the PUM or stakeholders, which can form the inputs 205 to the risk and safety systems. In some embodiments, these inputs 205 may be evaluated by the risk and safety systems 206, which generate one or more outputs 207. These outputs can, in some embodiments, be communicated one or more response systems 208, where one or more responses may be generated and, in some embodiments, may be communicated to an impact analysis system 209. The impact analysis system can receive or request one or more further data sets 211 from an input module, for example, receiving or requesting real time or near real time data sets generated by the one or more sensors, devices or systems 202 present in the SEE 204, including those worn or carried by the PUM. The impact analysis systems 209 can communicate with one or more action execution systems 210 to invoke actions or execute the appropriate instructions, commands or other specifications, including in the form of human interpretable data, to the one or more sensors, devices or systems 202 of the SEE or to the PUM 201 or stakeholders 203 present therein.

In some embodiments, evaluation of the one or more data sets or patterns representing one or more behaviors of a PUM can be used, at least in part to identify specific risks or safety issues. These identified issues can then be used, at least in part, for the calibration, configuration or operation of the one or more sensors, devices or systems, including those embedded in the environment or those carried or worn by the PUM.

These specifically targeted issues can form part of the HCP (Health Care Profile) of the PUM and may be used, in part or in whole, by the one or more care processing systems, including care hubs, care processing systems, game theory engines, AI/ML systems, digital twins, PPE, or any other systems involved in the monitoring of the PUM and the SEE.

For example, when a PUM undergoes a procedure such as a hip replacement or knee surgery, the one or more sensors, devices, or systems present in the SEE can be calibrated or configured to identify patterns that are more likely to occur after such a procedure. This evaluation can include, for example, the SEE scanning the environment for those elements within the environment that may represent an increased risk. For example, trip hazards, furniture, or other objects may have increased risk metrics in light of the procedure and any aftereffects, such as the impact of medications. This evaluation can include specifications provided by, for example, health care professionals, such as physical therapists, where certain movements, exercises, remedial or other activities are specified and using for example care processing or care hub, can be used to configure the one or more sensors, devices or systems to monitor for such occurrences.

There may be one or more classification schemes employed for the management of such monitoring targets, where these schemes, for example, can be arranged by procedure or injury type, severity, probability of occurrence, risk or safety metrics, behaviors or patterns, or the like.

In some embodiments, certain risks or safety metrics may be used to calibrate or configure the one or more sensors, devices or systems present in a SEE, including for example safety enabled devices, such that a PUM who exhibits particular behaviors can have specified risk or safety targets which, for example can be used to configure care hubs, care processing, risk and safety systems such that the PUM or other stakeholders or third part systems receive communications regarding such targets including, for example, predictions as to the probability of occurrence in one or more time frames.

This approach can be used, for example to prioritize the monitoring systems calibrations, configurations, operations or responses, in any arrangement. This approach can include the use of tokens to represent such data sets and in some embodiments, may be used to support the concatenation of complex data sets to simplified representations, including those that are human interpretable, such as for example those communicated to the PUM or other stakeholders.

One aspect of the risk management systems described herein is the representation of risk in the form of one or more sets of metrics. These sets of metrics can represent the measurement of the one or more risks of an environment, the actions or behaviors of a PUM domiciled or present in such environment or interactions of PUMs with one or more other stakeholders or external third parties. In some embodiments, there may be further risk metrics for those stakeholders who interact with the PUM in such environment.

In some embodiments such risk metrics are bound, for example, as attributes, parameters, variables or other characteristics, to one or more identified elements of an environment. For example, a set of stairs can have a higher risk metric than a flat surface or a flat surface that is wet can have a higher risk metric than one that is dry. These metrics can be defined as the baseline or inherent risk metrics of an environment, which can be incorporated into one or more risk calculations. For example, when a PUM is present or interacting with those environments and can, for example, be modified to represent dynamic changes in an environment, for example, using a multiplier.

Risk metrics can be attributes for, for example, one or more movements undertaken by a PUM. For example, rising from sitting to standing, walking, carrying things, showering, and the like. In some embodiments, such activities may be represented by mobility frameworks, which can be represented by, for example, a Personal Physics Engines (PPE), where each respective mobility framework can have one or more risk metric attributes.

In some embodiments, the sequence of such mobility frameworks (e.g., rising from sitting, traversing to a kitchen area and making a cup of coffee, etc.) can have an aggregate risk metrics representing this activity, including, for example, returning to the sitting position, and as such can be the risk metric for such a behavior. The aggregate risk analysis can include further risk metric variables, such as multipliers, where, for example, if this behavior is a regular behavior that occurs at a particular time of day (e.g., at or near mid-morning) then the multiplier may be less than one for this risk, whereas if this behavior occurs at a time that is not part of the regular behaviors, for example before dawn, then the multiplier may be greater than one.

In this example, further aspects of the environment risk metrics, can include, for example, trip hazards, balconies, stairs, slippery floors, loose fittings or other potential hazards, which can, at least in part be identified by the one or more sensors, devices or systems or by declaration, for example by a PUM or other stakeholder.

In some embodiments, risk and risk metrics may form representations of safety, where for example an environment, behavior, activity, event or other context or occurrence is safe for the stakeholders or environment involved. This description can be expressed in a number of forms, for example as a safety metric where the value of safety can be based on a number line, for example where neutral is zero, positive indicates a safe situation and negative indicates a reduction in safety, based upon a scalar.

The use of scalars and metrics as expressions of the degree to which an environment, behavior, activity, event, or other occurrence is safe can have an implicit or explicit context for such scalars and metrics. For example, if a PUM is walking from one location in the environment to another, this activity may have an expression of safety that is classified as safe, whereas if that PUM is walking outside the SEE, for example on an icy path to a letterbox, this activity may have a low safety metric and be classified as unsafe. Both the safe and unsafe classifications can have a bound metric, for example from −10 to +10, to represent the severity of the situation.

The use of classifications, such as safe, neutral, unsafe, which can be determined, in part or in whole, by one or more metrics and scalars, enables the communication of the appropriate granularity to, for example, a PUM or other stakeholder, whilst informing, for example, one or more sensors, devices, or systems of the underlying metrics involved, such that one or more evaluations, processes, or calculations may be undertaken by such sensors, devices or systems, including, for example, care hubs or care processing systems, including those employing AI/ML capabilities. The communication to the sensors, devices or systems can include the calibration, configuration or operations of such sensors, devices or systems. For example, the PUM may be sent a message such as “slippery surface ahead”, whereas the underlying metrics can include one or more algorithms or data sets.

In many circumstances, the determination of safety can be, in part or in whole, contextual. For example, the condition of the PUM can be a significant influence on this determination. For example, if the PUM has limitations to mobility, for example, arthritis, knee damage, and the like, then walking up a steep incline may be determined to be unsafe for that PUM at that time. In contrast, that same activity may be determined to be safe for a different PUM that does not have mobility limitations.

The same approach may apply to locations, for example, where a PUM in one location, for example on a carpeted floor, the PUM can be determined to be able to safely traverse that floor, whereas if the floor is tile and is wet, the PUM may be determined to not be able to safely traverse that situation, despite partaking in the same underling action of “walking”. This location-based determination can include, for example, the identification of environmental hazards, such as an exposed electrical outlet where the wiring is accessible, poorly secured hand or guard rails, uneven floors, and the like that can be described as unsafe. These classifications can have one or more metrics or scalars that are bound to the classification, each of which may have a correlated message that can be provided to the PUM or another stakeholder. For example, if the slippery floor has an unsafe categorization for a specific PUM at that time, and for example a severity of 7, where 10 is most severe, there can be a correlated message, for example “do not proceed/high fall risk” which is communicated to the PUM or another stakeholder. In this example, the other stakeholder may be a carer, to which a further message is communicated, for example “assist PUM with potential fall hazard”.

In addition to personal details and location details associated with a safety context, the safety of a context may in part depend on the timing of a PUM's behavior, for example, in low light circumstances, or at irregular timing for that activity the safety metrics of a particular behavior or action may vary according to that timing. For example, a behavior historically performed during daylight hours (e.g., preparing a meal) may be determined to have a first safety and context, but when the same behavior is identified as being performed in the middle of the night, the behavior may be determined to have a second safety and context rating to indicate a higher likelihood of being hazardous (e.g., due to the PUM being disoriented or confused or secretive, due to different lighting conditions, due to the presence or absence of other stakeholders in the SEE compared to the typical historical timing, etc.).

In some embodiments, one or more sensors, devices or systems may be configured that upon certain patterns of data being detected, for example, at a specific location or at a specific time period, these patterns may be evaluated, by for example a risk evaluation system, care hub or care processing system in any arrangement and consequently have a risk or safety metric and appropriate classification bound to that pattern. These patterns and bound safety or risk metrics or classifications may be represented as one or more tokens, and in some embodiments such tokens may be communicated to, for example one or more sensors, devices or systems, care hubs or care processing systems, risk or response systems, AI/ML systems, game theory engines or stakeholders, including the PUM in any arrangement. The use of tokens can include, in some embodiments, tokens that have separate segments with differing data sets for specific recipients. For example, a first segment can comprise a warning to the PUM, for example “fall hazard ahead”, whereas a second segment can comprise configuration data for the one or more sensors, devices or systems worn or carried by the PUM. A third segment can comprise a further message to one or more carer or other stakeholder. Each of the parties may receive the same token, but is only permitted to access the segment of data in the token associated with that party (e.g., via different encryption schemes applied to the different segments) to thereby allow the token to be forwarded or shared among the parties without compromising data security.

One aspect of this approach may be the timely detection of any context that can adversely impact a PUM, such that as far as possible in advance this (potential) adverse context can be communicated to the PUM, or other stakeholders, so that the potential impact may be avoided or mitigated to the degree possible.

In some embodiments, these metrics of risks for patterns, behaviors, locations, times, interactions or other actions, occurrences or events may be evaluated to, in whole or in part, represent the safety of the situation. For example, if a set of patterns have a set of risks and such patterns form a behavior, then that set can represent the overall risk of the behavior. If this behavior occurs at or within a time period or at a particular location, these time and location factors may contribute to the risk for that behavior at that time at that location. Such a risk measurement may then be aligned with one or more safety scalars, for example ranging from unsafe to safe or dangerous to beneficial. These safety evaluations can then be communicated to a PUM or other stakeholders in any arrangement.

Such safety metrics can be used for one or more calibrations, configurations or calculations by the one or more sensors, devices, or systems of a SEE, including for example care hubs or care processing systems, which can include, by reference or embedding one or more AI/ML systems.

In some embodiments, such safety metrics may be communicated to one or more stakeholders, including the PUM to inform the stakeholders that a particular behavior has a safety metric. For example, these communicated safety metrics can include safety metrics that are predicted as well as those calculated in real time. One advantage of this approach may be the reduction of potentially complex risk measurements to quantized human readable and understandable communications, for the benefit of one or more stakeholders including to the PUM.

In some embodiments, one or more AI/ML systems, including one or more LLM or LCM may be employed to determine, in part or in whole, such safety metrics, where for example risk metrics can form part of a data set and the safety metrics are determined, at least in part, by a classification schema based at least in part on these risk metrics.

In some embodiments, safety metrics can include expressions of safety by the one or more stakeholders, for example a PUM may declare that they are unsafe or that in undertaking a particular behavior they have concern for their safety. In some embodiments, one or more sensors, devices or systems may be configured to recognize, using for example audio (including Natural Language Processing (NLP)), haptic, visual, gesture, biometric, physiological or other communications from the PUM that such a safety situation is perceived by that stakeholder.

In some embodiments, the one or more sensors, devices or systems of the SEE can be employed to validate, verify or evaluate the current or predicted situation of the PUM, where a PUM has indicated a safety concern. When the data sets of sensors, devices or systems and the risk or safety metrics thereof correlate with the perceived or declared state of the PUM, one or more communications, including to one or more care processing systems or care hubs, may be generated which can then result in, for example, communication with the PUM, other stakeholders, including for example carers, emergency services, or the like. For example, the communications can include warnings, such as “watch out for the cat” and the like. In some embodiments, one or more indicators of the risk, for example, a smart light bulb may activated to identify the actual or potential risk, for example, an obstacle or change in condition of a surface, for example from dry to wet.

In some embodiments, safety expressions may be communicated to the one or more PUM or other stakeholders in an environment, where, for example, such communications may be provided in advance or in near real time to the PUM or other stakeholders. This provision can include communications that represent simplifications, including colloquialisms of the potential risk or safety metrics, where the focus of such communications is the effectiveness of those communications in providing the PUM or other stakeholders with appropriate and useful human interpretable data. Accordingly, a safety expression may be represented using terminology matched to the reliever to convey an underlying concept with a requisite level of detail or simplicity in terminology. For example, a safety expression for a potential fall event may be communication to a PUM who is predicted to fall using short, direct wording (e.g., “Caution! Tripping Alert!”), whereas a safety expression for the same prediction is provided to an in-home carer for the PUM using directed wording with greater detail to quickly direct aid for the PUM (e.g., “Alert! PUM in kitchen needs assistance”), whereas a safety expression for the same prediction provided to a remote stakeholder (e.g., a friend, family member, designated contact, emergency responder, physician/medical practitioner, etc.) is provided with diagnostic detail in greater detail (e.g., “PUM exposed to potential tripping hazard when moving from living area to kitchen. Hazard identified as dog sleeping in doorway”).

In some circumstances, various combinations of environment and actions can equate to differing states of safety which can be measured using the one or more sensors, devices, or systems in a SEE and may be perceived by a PUM or other stakeholder. In some examples, the measured circumstances may align closely with the PUM perception, for example where the PUM directly or indirectly expresses their perceived safety. For example, a PUM may indicate through the one or more sensors, devices, or systems that they feel safe or unsafe, including the degree thereof. For example, a PUM may state “I feel unsafe” in particular circumstances, which can be received by the one or more sensors, devices or systems with audio capture and analysis capabilities, for example a suitably configured smart phone, embedded one or more microphones, personal emergency response system (PERS) devices, or the like. Theses declarations may include a range of phrases, for example “I am worried”, “I am scared” and the like, which can be interpreted, for example using speech to text and NLP processing and can consequently be correlated to one or more safety or risk metrics.

In some embodiments, such safety concerns may be detected by the one or more sensors, devices or systems present in the SEE. This detection can include the use of, for example, worn or carried devices, where for example heart rate, sweat, breathing rate and type, eye movement, or other physiological characteristics may be measured and correlated to PUM safety or risk metrics. This usage can include, for example the one or more sensors, devices or system present in the SEE detecting, for example, hesitation of a PUM in a particular behavior, which can indicate the concerns of the PUM for their safety.

These PUM or other stakeholder safety perceptions can be compared with the measured safety or risk metrics of the current situation to ascertain any alignment or correlation, including the degree thereof. These evaluations may further inform, for example, one or more care hubs or care processing systems as to the PUM safety, including where the measured safety and risk metrics are low, yet the PUM perception is a concern for their own safety, and as such one or more communications may be generated for this situation, for example to a carer, relative, third-party service or the like. In some situations, this comparison and evaluation may further indicate a change or variance of the physical or mental state of the PUM and may inform one or more care processing systems including care hubs of this variance.

In many circumstances, the self-declarations of a PUM may be absent, in that the PUM makes no explicit statements as to their safety, in which case the self-declared safety metrics may be null; however, the one or more sensors, devices, or systems, including those worn or carried, may provide data sets that have safety or risk metrics that indicate an actual or potential safety concern. In this example, such safety or risk may be communicated to one to more stakeholder, including the PUM.

In some embodiments, one or more safety and risk metrics may include leading or lagging measures of a PUM behaviors, which can include predictive measures, based at least in part on sparse data sets from the one or more sensors, devices or systems of a SEE, where, for example one or more best fit algorithms can be used to determine the potential patterns or behaviors and their one or more safety or risk metrics. In some embodiments, one or more AI/ML systems may be employed in the prediction of such measures.

These predictions can include, for example, evaluation of activities of one or more PUM. For example, a set of PUM who have similar conditions or situations may be identified and the data sets representing the patterns or behaviors and the safety and risk metrics of those PUM can be aggregated and, in some embodiments, may be used by one or more predictive systems, including AI/ML, digital twins or game theory engines to predict safety and risk metrics for a specific PUM.

In some embodiments, the environmental or locational based risk metrics, (e.g., of wet areas, food preparation areas such as kitchens, areas with uneven surfaces or differing surface heights, or areas containing furniture, fittings, or objects or the like) can have differing risk or safety metrics that represent the contributions of those locations to any potential risks for a PUM when the PUM is in those areas. This evaluation can be, for example, a variation on any constant or baseline that is used for calculating such risk metrics, and can include further variations to account for the time periods when the PUM may be present in those locations. In some embodiments, this evaluation can include further variations based on, for example, ambient or direct lighting, floor surfaces, including if there is any wet patches and the like.

In some embodiments, the metrics of risk may include a triple, for example, including metric expressions for health, wellness or safety, where each of these expressions includes at least one category/value pair.

In some embodiments, risk or safety metrics may be perceived by different stakeholders as representing different states of risk or safety. For example, a first stakeholder may find a specific risk metric (RM1) as exceeding their perceived risk threshold, and consequently may react to that risk, whereas a second stakeholder may perceive the same risk metric (RM1) as falling below their risk threshold and consequently consider the situation to be sufficiently safe as to not warrant any action on the behalf of the second stakeholder or anyone else. These risk thresholds can form part of an HCP for a PUM or may form part of the profile of a stakeholder (e.g., a non-PUM stakeholder).

In some embodiments, one or more AI/ML systems in conjunction with the one or more sensors, devices or system of the SEE may deploy or generate one or more risk or safety thresholds for the one or more PUM or other stakeholders. These risk or safety thresholds may include, for example, the state of the environment, locations therein, time period, or the one or more patterns or behaviors of the one or more PUM or other stakeholders.

These risk or safety thresholds can, at least in part, inform the one or more stakeholders, including the PUM, as to the perceived risk or safety of a situation and communicate these risk or safety metrics, including summaries thereof, to one or more other stakeholders. For example, risk metrics or safety metrics, including the personalized thresholds of a PUM, may be used to select or align a carer or other stakeholder, using the profile of that stakeholder for those metrics, to match those of the PUM or, in a particular monitoring situation, for example where the PUM has undergone a procedure or experienced an event, such as heart attack and the like, these criteria may also be used.

In some embodiments, these risk or safety thresholds may form part of a risk or safety profile for a PUM, other stakeholders, an environment, or locations therein, one or more time periods, one or more patterns or behaviors or the like. In some embodiments, one or more AI/ML systems, including one or more LLM or LCM, may be employed to predict the one or more risk thresholds of environments, locations, time periods, patterns, behaviors, or other measured situations, which can then inform the one or more perceptions of such risk or safety thresholds of the one or more PUM or stakeholders.

In some embodiments, one or more digital twins, including those of the environment, representing the one or more sensors, devices or systems present therein and those representing the PUM, for example one or more digital twin incorporating a PPE, can be configured to represent the current or predicted behaviors of the PUM or other stakeholders in such environment.

In some embodiments, digital twin based simulations may include one or more PPE as representations of the PUM. These PPE may be integrated into one or more digital twins of the environment, including the SEE and the one or more sensors, devices, or systems, including care hubs and care processing systems therein and any other stakeholders present or anticipated to be present in the environment. In some embodiments, the PPE may form part of a digital twin of the PUM, in whole or in part.

Such digital twin arrangements may be used to, at least in part, predict PUM behaviors, using for example one or more AI/ML system. These predictions may be based, at least in part, on the behaviors of the PUM, where measurements from the one or more sensors, devices or systems provide data sets, which may be in the form of patterns, which can form the one or more behaviors, that can be used to inform such simulations.

One aspect of these simulations may be the use of predictions to anticipate a set of patterns or behaviors that can indicate, at least in part a potential health, wellness or safety impact on a PUM. This predictions can include anticipating the safety of the PUM, where for example a potential impact may include one or more safety metrics.

In some embodiments, safety or risk metrics may be calculated based on one or more sequence of patterns, mobility frameworks or behaviors. The calculations can include, for example, risk or safety metrics that are based, at least in part, on location or time of day.

Currently, there are many techniques for calculating risk in a wide range of circumstances and situations, including for example, Bow Tie, Delphi, SWIFT, Probability/consequence matrix, Decision tree analysis, financial risk models such as Alpha, Beta R squared, standard deviation, Sharpe ratio and Option models (Black-Shoals) as well as risk matrices including decision risk matrix assessment (DRMA) which are used in health care.

Predominately these current models use statistical approaches for determining risk, often using a top-down approach, rather than a sensor, device, or system data-based approach, where the risks can be considered from a bottom-up perspective. These sensors, devices, or systems data approaches as embodied in a SEE can provide a more accurate, timely, and relevant risk or safety evaluation model that can be configured for the particular PUM in a specific environment over one or more time periods. This approach can include, in some embodiments hyper-personalization of risk or safety metrics, AI/ML models, responses or actions as the one or more sensors, devices or systems, in collaboration with one or more AI/ML systems that learn the behaviors of the PUM and the proclivities, actions, activities or other ephemera of the lived experience of the PUM.

In some embodiments the evaluation or simulation of risk can involve one or more game theory modules, where the actions of the PUM can form part of the game to determine, at least in part the possible risks and corresponding strategies for those addressing those risks and game outcomes. This evaluation or simulation can include configuration of one or more game theory modules to represent a particular PUM in a specific set of circumstances, which can enable a more timely and accurate risk simulation and evaluation.

The combination of game theory modules and AI/ML systems provides an effective approach to determining the safety and risk possibilities based, for example, on sparse input data sets such as a data sets generated by the one or more sensors, devices, or system of a SEE, where for example such data sets can include one or more exceeded thresholds, representing for example an edge condition that indicates a change from a quiescent to non-quiescent state for the environment.

In some embodiments, a Personal Physics Engine (PPE) may be used to represent the actual or potential movements of a PUM is a SEE. For example, a PPE may be employed in one or more digital twins of the environment and can represent the movements of the PUM therein. In this manner, actual or predicted movements of a PUM and the risk or safety metrics of the PUM can be evaluated.

For example, using the behaviors of the PUM, including those that are related to specific locations or time periods, one or more PPE and digital twin combinations can be used to generate representations of the future behaviors that involve mobility to evaluate the risk or safety metrics of those behaviors. This evaluation can be used, for example, to calibrate, configure, or operate one or more sensors, devices, or systems of the SEE or can be used, in whole or in part to generate one or more responses, including communicating with the PUM or other stakeholders regarding such anticipated behaviors.

In some embodiments, there can be one or more arrangements of PUM patterns or behaviors and the risk or safety metrics for the PUM, for example as an ontology, taxonomy, or other organization, such as a directed or acyclic graphs. These arrangements can be used, at least in part, to inform sensors, devices, systems, or stakeholders of these relationships, for example as a communication.

In some embodiments, the outputs of these simulations may be further evaluated, for example using one or more AI/ML system, to ascertain potential further behaviors or patterns, such as those that can occur as one or more characteristics of a PUM vary, for example, deteriorates over time. For example, as mobility, eyesight, mental acuity, and the like can degenerate over an extended period, such evaluations may represent the potential characteristics of the PUM or other stakeholders at a future time.

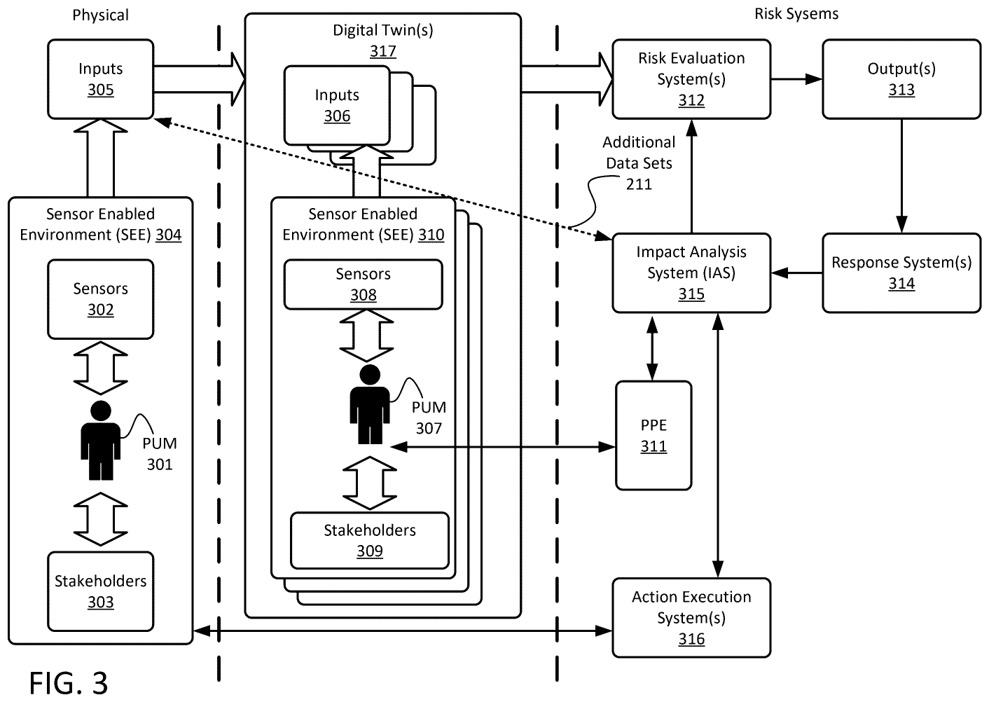

FIG. 3 illustrates an example embodiment where, in the physical domain a PUM 301, other stakeholders 303 and one or more sensors, devices or systems 302 are present in a SEE 304. The one or more sensors, devices or systems 302, PUM 301 or other stakeholders 303 can generate actions and data sets that can form the inputs 305 for one or more predictive or risk systems. In some embodiments, the physical SEE 304 and the contents thereof can be represented by one or more digital twins 317, which can include digital twins of SEE 310, PUM 307, sensors, devices and systems 308, stakeholders 309 and the one or more inputs 306. In some embodiments, these digital twins can be used to, at least in part, predict the activities, operations or data sets of the physical SEE 304, PUM 301, sensors, devices or sensors 302 or stakeholders 303 and the representations thereof as inputs 305 to one or more other systems, such as risk systems, in any arrangement. This digital twin approach can include the use of one or more PPE 311 to represent, at least in part, the movements and mobility of the PUM 307. In some embodiments, one or more risk systems 312 can evaluate the inputs 305 for the physical domain and those of the digital twins 306 in any arrangement. The risk systems can generate outputs 313, which can be communicated to other systems, including response systems 314, although these outputs can be communicated to the one or more digital twins. The response systems can communicate with the impact analysis systems 315, which may receive or request data sets for the inputs 305. The impact analysis systems 315 can communicate with PPE 311 or action execution systems 316 in any arrangement. The action execution systems 316 can communicate with the digital twins 317 and the elements thereof or the physical SEE 304 and the elements thereof in any arrangement.

In the domain of health, wellness and safety, there is no one size fits all approach. Many current systems employ statistical models to determine such factors. Although these models may provide some indication as to the condition of the PUM, the use of personalized metrics for these factors supports a greater granularity of detection or prediction of any current or potential neutral, adverse or remedial circumstances. These improvements include the avoidance of false positives and the detection in advance of any likely health, wellness or safety concerns.

In some embodiments, one or more knowledge bases may be instantiated, including for each of the individual PUMs and for an aggregated set or population of PUMs. Such knowledge bases can include, for example, data sets from the one or more sensors, devices or systems of a SEE, patterns, tokens or behaviors from such or from one or more care hubs or care processing systems or one or more predictive systems, including those incorporating one or more AI/ML systems, game theory systems, digital twins, risk evaluation systems or response systems in any arrangement.

In some embodiments, those personalized characteristics for a PUM may be anonymized and contributed to one or more knowledge bases or other repositories to create a generalized model for one or more population of PUM. For example, anonymization can include the use of tokens where the origination of the token is a one-way function.

For example, in some embodiments, a knowledge base may include location, time, behavior, and context data in any arrangement. For example, such a knowledge base may be implemented using, for example, graph databases, structured databases, ontologies, taxonomies, lattices, matrices, including for example neural network weight matrices, manifolds, or other topological arrangements and the like.

In some embodiments, such knowledge bases may be configured to enable one or more stakeholders or systems to invoke a representation of the data therein which can represent, for example, a stakeholder or a proxy for a stakeholder, including one or more devices or systems the stakeholder or proxy control or have authenticated access to, a perspective of the stakeholder or proxy in and of a particular situation or context.

For example, a behavior can include one or more patterns that are representations of data sets generated by the one or more sensors, devices or systems present in a SEE. These behaviors may be bound to one or more stakeholders, typically the PUM, however there may be behaviors bound to other stakeholders, such as a carer or to multiple stakeholders, such as when a PUM and carer interact.

Each of these behaviors may have one or more risk or safety metrics which form part of the behavior and, as such, can be evaluated by the one or more systems deployed in a SEE. Such metrics may, for example, form attributes of such behaviors and be represented in a repository which can, for example, be an instance of a knowledge base.

Similarly, environment locations, including those internal to the environment and external to the environment, may also include such metrics, and can, for example, be represented in a knowledge base or other repository.

In some embodiments, time, expressed, for example, as time of day, may also be represented in such a repository, where, for example, such clock time may be expressed both in the form of a 24-hour clock or in the form of time periods representing, for example, the one or more experiences of a PUM, such as, for example, sleep, lunch, breakfast, exercise, a therapy session or the like.

In this manner, the various relationships between the one or more entities stored in the knowledge base repository may be determined so as to establish, at least in part, the combined risk and safety metrics for a specific situation which involves, for example, one or more behaviors, activities, events or other recognizable occurrences, at a location and a time.

Establishing risk and safety metrics for time and location can, in some embodiments, enable risk and safety profiles and data sets where known behaviors have outcomes with various risk factors for a PUM or other stakeholders. In some embodiments, such risk factors may be represented by one or more vectors or multi-dimensional feature sets.

In some embodiments, this approach can enable the one or more sensors, devices or systems employed in a SEE, which are generating data sets or patterns representing, at least in part, these behaviors, to represent these behaviors or underlying data as tokens, that can include the identification of a change in state of the PUM behavior or environment, such that at the earliest observable instance of such variation within the data sets can be evaluated, through for example, evaluation of the one or more risk or safety metrics bound to these data sets or the data sets themselves, to identify any potential or actual increase in the risk or safety of or to a PUM, and potentially other stakeholders.

One aspect of this approach may be the identification of one or more behaviors where, although the data sets or patterns generated by the one or more sensors, devices or systems correspond to a known behavior, the data sets deviate from an initial correlation. For example, a PUM may start a behavior, such as walking to the kitchen, and then retrace their steps or walk into another room. In this example, the uncompleted initial behavior may be represented by a different set of risk or safety metrics as may the combination of the partially completed initial behavior and subsequent behavior.

The identification of the start and end of a behavior can include, in combination with other contextual data, time-based frameworks for the initiation and completion of such behavior. These time period data sets may be stored in one or more repository, including knowledge bases, which can then support the evaluation of these time periods when, for example, one or more thresholds are exceeded.

In some embodiments, these time periods for a behavior can have risk or safety metrics bound to them. For example, if a behavior takes a time period outside of the normalized time based distribution for that activity, then the risk or safety metrics may increase.

In some embodiments, as the known behaviors of a PUM become established, for example, eating, sleeping, exercising or performing other regular activities at particular times, these behaviors can comprise a set of patterns or behaviors from initiation to conclusion. For example, a making lunch behavior may comprise a set of patterns that represent the activities, including mobility, of the PUM, which can be represented by a set of patterns in a particular sequence equating to the making lunch behavior.

Such a sequence can have certain dependencies, for example, sourcing the materials for the lunch is a predicate to preparing those materials for consumption. In the broadest sense, initiating a behavior is a predicate to completing the behavior.

Such sequences of patterns or behaviors, can provide an indication of the intention of a PUM, in that if a sequence is started and completed on a regular basis, for example as a daily routine, then any divergence from that routine can indicate an elevated potential risk or safety issue.

In some embodiments, where a sequence has been initiated and then not completed, the interrupted sequence can indicate that the mental acuity of the PUM may have deteriorated. For example, if step (X) in a sequence is making a sandwich and step (X+1) is returning the materials to the refrigerator, and step (X+1) is not undertaken in a single occurrence, then the sensors, devices or systems, including risk and safety enabled devices or applications, may prompt the PUM to undertake step (X+1).

However, if the sequence of steps becomes erratic or has a consistent characteristic of uncompleted behaviors, including the sequences thereof, these interruptions to the sequence can indicate an elevated risk or safety concern, which can be communicated to, for example the PUM, for example as a prompt or reminder, to one or more stakeholders, for example a carer, or to one or more sensors, devices or systems. In the latter case, communication to one or more sensors, device or systems can include the calibration, configuration or operation of such sensors, devices or systems.

In some embodiments, such sequences can have risk or safety metrics which can form, for example, a risk or safety profile for the behaviors being undertaken. For example, the risk or safety metrics may be higher at that point in a behavior sequence, for example making lunch, when the PUM has a sharp knife, than when the PUM returns the materials to the refrigerator.

For example, in some embodiments, sequences of risks may be evaluated as 1/(risk1+risk2) or risk1+risk2, where risk1 and risk2 are respectively the risks for step1 and step2 in a sequence where step1 precedes step2. These risk sequences may be used, for example, as training data for one or more AI/ML systems. For example, such an approach may be used to generate further metrics or classification for risks or safety, for example competence. In some embodiments an RNN may be employed as part of an AI/ML system to evaluate these steps, and can include the use, for example, of RBM, to represent the risks, expressed as metrics, which can, in whole or in part, determine the weightings used by the RNN, where the RNN can predict the next step in a sequence. For example, in many circumstances, a behavior may comprise a set of patterns, each pattern of which can include one or more steps or tasks which typically are completed in an order. However, in some circumstances, the order of the steps may vary, for example first slicing bread, then slicing cheese versus first cutting the cheese and then cutting the bread, and as such although the order of the steps or tasks varies, the overall intention and execution of the pattern is consistent with the behavior. In this manner, false positives can be avoided and the variations in such behaviors can form part of the model of the behaviors, where for example the potential range of variations is correlated, such that any inconsistencies with the overall intent and execution of the behavior can be identified at the earliest possible time.

The use of risk sequences can, in some embodiments, enable the early detection of potential health, wellness or safety impacts for a PUM. For example if a particular behavior is initiated, and such behavior has a particular risk profile, for example R1, R2, R3, R4 . . . . RN, where R is a risk metric, and the one or more sensors, devices or systems that detect that a pattern is forming such behavior having such a risk metric that varies from the anticipated risk profile, this analysis can indicate an elevated risk to the PUM, which can result in a response including for example, an alert, prompt or other communication being sent to the PUM, other stakeholders or sensors, devices or systems in any arrangement, including for example risk and safety enabled devices or applications.

These risk sequences may be aggregated across multiple PUMs to create risk profiles for sets of behaviors, where the sequence of patterns making up those behaviors and the risk metrics may differ, yet the overall behavior, for example making lunch, has a consistent initiation, duration and conclusion.

In some embodiments, there may be contextual risks that are related to one or more known or identified locations or location types, with one or more correlations to neutral, negative or positive impacts for one or more PUM. Such impacts can include both physical impact, such as when a PUM stumbles or trips or mental impact where for example a PUM gains pleasure or exhibits fear for their safety and the like.

In some embodiments, risk for each data set, pattern or behavior may be represented as a constant. This constant, for example K, may be applied to data sets, patterns or behaviors where these data sets are within the configured thresholds of these data sets, patterns and behaviors or are part of the quiescent state for the PUM or environment. This approach simplifies the degree of configuration for such patterns or behaviors whilst providing a risk metric for the patterns or behaviors. A similar approach can be undertaken for safety metrics, where a simplification of such metrics using a constant, for example neutral, safe or unsafe can be bound to one or more behaviors.

If a data set generated by one or more sensors, devices or systems within the SEE has one or more measurements that are outside the calibrated or configured thresholds for that data set, for example, a pattern representing movement, such as a mobility framework, then the risk metric, may be varied from the constant (K), by a variable that is aligned to the degree of variance of the threshold, a quantized variable representing this variance or a further indicator of the variance.

For example, the constant (K) may be represented on a number line, where in quiescent circumstances, K=1 and the number line ranges from 1 to −1, such that deviations that have limited potential for impact on the safety, wellness and health of the PUM are in the range of 1 to zero, whereas any negative value indicates a potential negative impact on the safety, wellness or health of the PUM. There may be other scalars, including logarithmic, exponential, linear or other expressions of such constants.

In some embodiments, various constants may be used for each data set, pattern or behavior that is measured by the one or more sensors, devices or systems of a SEE. Each of these constants may represent the relative risks for those patterns or behaviors, individually or in aggregate. For example, a sequence of patterns, for example a set of movements represented by movement frameworks, may have a set of risks represented as a set of constants, such that each of these constants can, in aggregate, represent the risk of this sequence of mobility frameworks as long as the data sets for these mobility frameworks are within the configured thresholds. Should, for example, a data set have one or more measurements that exceeds the configured threshold, a risk evaluation system or other monitoring systems, such as a care hub or care processing system, may then calculate or assign a different risk metric to that data set. This action can then result in the recalculation of the aggregate risk for that mobility framework or may include invoking one or more other systems to create an alert or communication to, for example, a PUM, other stakeholder or one or more sensors, devices or systems.

There can be one or more behaviors or patterns that can indicate the condition of a PUM, for example, a personal hygiene routine (including lack thereof), social interactions (including lack thereof), mental resilience, diet, mobility and the like. In some embodiments, these observed and measured behaviors may provide indications as to the overall stress level of the PUM. These stress levels can contribute to the risk or safety profile and the metrics of a PUM.

In some embodiments, one or more AI/ML systems, including one or more LLM or LCM may be employed to determine, at least in part, the intention or attention of the PUM, through evaluation of the data sets, patterns and behaviors of a PUM. This evaluation can, in some embodiments, include data sets generated by one or more worn or carried sensors, devices or systems, which can include biometric and physiological sensing capabilities and the data sets generated by the one or more sensors, devices or systems of the SEE. In this example, the sets of data may be evaluated, using for example an LLM or LCM, to ascertain the intention or attention of the PUM.

In some embodiments, one or more risk metrics may form one or more safety metrics in that the set of risk metrics for a PUM, for example, those of the environment, location, time period, movements or behavior of the PUM at that location in that time period can have an overall safety metric. Such a metric may be communicated to the PUM or other stakeholders, for example, if the one or more sensors, devices or systems present in the SEE monitors the state of the SEE to be quiescent, then such a safety metric can, for example, be determined as safe for that PUM in those circumstances. Although such a safety metric indicates that the current situation is safe, the metric can represent the absence of measured or observed risks, actual or potential at that time, rather than any surety of the safety of the PUM. For example, such a safety metric expressing that the situation is safe, can indicate the lack of measurable or anticipated risks such that the risk metrics of the measured situation are below any thresholds, for example those of a quiescent state or the predicted risks have probabilities that are sufficiently low as to be unlikely to change that state in the immediate, foreseeable or calculated future.

In some embodiments, safety can be contextual, in that there is the perception of safety, which can be subjective, as experienced by a PUM or other stakeholders and the degree to which a situation, circumstances or an environment can cause, in whole or on part, that perception to change.

Many existing systems employ safety metrics, focused on incidents and the reporting thereof, where the intention is to catalog those incidents and the potentially the causes thereof. In the context of a PUM, where that PUM has an HCP and is primarily domiciled in a SEE, safety evaluation can include identification of those risks that may impact the safety of that PUM, and in part to reconcile the PUM's perception of their safety with their observable behaviors or context to ascertain a contextual perspective on their safety.

The role of prediction of a PUM's behaviors, including both physical dimensions, for example, those predicted by a PPE in a digital twin, and any mental dimensions, where the PUM perception may not match the observable data, can provide data sets that may be used to calibrate or configure one or more sensors, devices or systems, including care hubs or care processing systems to, for example communicate with the PUM or other stakeholders to, at least in part, mitigate, remedy or avoid any health, wellness or safety impacts.

One aspect of risk and safety identification and reconciliation is balancing the potential risk with any observed risk. For example, if a PUM trips over a carpet or pet, there are a number of potential outcomes, each with a risk or safety metrics that represent such outcomes occurring. For example, if the PUM falls, this event has a number of possible outcomes, ranging from serious, such as broken hip, head injury or another life-threatening situation, to minor, such as the PUM regaining balance and possibly stubbing a toe.

The identification of any risks can be, at least in part, determined by the measurements represented by the data from the one or more sensors, devices or systems present in the SEE. In some embodiments, when one or more sensors, detect an edge condition, that is a condition that changes the quiescent state, exceeds one or more thresholds, has a vector that has a trajectory to exceed one or more thresholds or has one or more values that represent an edge zone, such as the initial change in orientation of the PUM, such as when the PUM trips over an object and, for example, an accelerometer detects an increase in acceleration of the upper body, a visual sensor capturing the vertical orientation of the PUM or an audio or haptic sensor detecting a noise or impact, these sensors can provide these data to one or more systems, including risk identification systems.

These risk identification systems can then evaluate these data to generate a set of risk or safety metrics that correspond to the risks of a set of outcomes based, at least in part, on the data set from the one or more sensors, devices or systems.

In some embodiments, these outcomes may all have an initial data set generated by the one or more sensors, devices or systems, for example a pattern indicating a trip or fall or a mobility framework indicating a trip or fall. This initial dataset, pattern or mobility framework may initiate one or more risk identification systems to generate a set of risk metrics for the one or more outcomes that can be a consequence of this initial detection. For example, the outcomes can include a set of graduated results, based on the severity of the outcomes and the probabilities of those outcomes occurring.

In some embodiments, one or more knowledge bases may be used to ascertain the potential outcomes. Ascertaining the potential outcomes can include, for example, where such knowledge base has a repository of data sets, patterns, mobility frameworks or behaviors that can be initiated by the originating detection. For example, such a knowledge base may be in the form of one or more ontology, taxonomy, graph database or other organization, such that the potential outcome may be determined and the risk metrics thereof configured, calculated or retrieved.

In some embodiments, the use of PPE in combination with one or more digital twins can provide mobility-based sets of simulated actual or predicted movements of a PUM in a SEE. This approach can generate simulated movement models which are based on movement frameworks configured to represent the measured movements of the PUM. This bottom-up approach to building more comprehensive movement patterns or behaviors can be used to create simulations of the PUM that are consistent with the actual physical capabilities of the PUM.

Each of these mobility models incorporating the movement frameworks can have risk or safety metrics which are the aggregate of the risk or safety metrics of each of the individual mobility frameworks. These risk or safety metrics can be used, in whole in part, to calibrate, configure or provide operating data to one or more sensors, devices or systems or other systems.

In some embodiments, each of the mobility frameworks represented by a PPE can have a range of risk or safety metrics, for example, expressed as a set with minimum and maximum values. This representation can include one or more functions, for example the range may be expressed as an integral with minimum and maximum boundaries or one or more weightings or further algorithms or functions.

The use of a PPE may include the determination, in whole or in part, of the potential mobility frameworks that can be undertaken based on the current or anticipated position of a PUM as represented by the PPE. In this manner, potential sequences of movements of the PUM can be evaluated.

In some embodiments, each movement framework can have commensurate risk or safety metrics, which can be represented as a function that, for example, includes a constant, representing the inherent risk metrics, a time, representing the time at which such movement is initiated, a duration, representing the period over which such movement is undertaken, a completion, representing the completion of the movement or a locational risk metric, representing the location at which the movement was undertaken.

In some embodiments, such movement risk metrics may include, for example, movement frameworks or modules having risk weighting based, at least in part, on sphere of interaction, where the sphere or interaction is representing the range or potential interactions a PUM can have in undertaking a movement. For example, if the movement is walking, this representation could include the range of steps and the surfaces such steps may interact with or the range of reach of the arms of the PUM that can contact any surfaces, furniture, fittings or objects, which can include other stakeholders.

In some embodiments, the one or more sequences of patterns or behaviors including the movements thereof, may form one or more predictions of the movements of the PUM, for example using one or more PPE or digital twins.

One indicator of risk is the degree of attention or focus a PUM or other stakeholder is applying to an activity, task or movement being undertaken. Measuring the degree of attention that is being applied by a PUM or other stakeholder can provide data sets that can, at least in part, contribute to or inform the one or more risk or safety metrics for that activity, task or movement.

In some embodiments, one or more sensors, devices or systems can measure the movements and activities of a PUM or other stakeholder, including for example, the direction or field of vision or the one or more likely focus points of that vision. The measurement of direction field or focus of vision can include the use of one or more simulations involving, for example a PPE, digital twins or one or more AI/ML systems to determine, at least in part, the potential attention or focus of that PUM or stakeholder.

For example, the direction of the PUM vision can be ascertained by, for example, a worn or carried device that is forward looking, for example, a lapel sensor, pendant or other worn sensor that includes one or more camera or other visual sensor. In some embodiments, such a device may also include LIDAR, RADAR, LASER, or other emission-based technologies to identify the potential focus points of the vision of the PUM or stakeholder.

This simulated or actual set of focal points can be correlated to the potential attention of the PUM, and can include the probability of that focal point being the main attention point of the PUM or stakeholder. As attention is core to the cognitive awareness of the PUM or stakeholder in a particular situation, the degree of attention to the environment and the potential risks thereof and the attention of the PUM or other stakeholder to those risks can provide data sets that inform the overall potential risk. These overall risks may, in some embodiments, be communicated to a PUM or the stakeholder, for example through haptic, visual, text, audio or other communications means, to alert them of the potential for increased risk and potentially to have them refocus attention on those potential risks. In some embodiments, this refocusing can include varying one or more environmental systems, for example lighting, window fittings, TV or other fixed appliances, audio systems and the like to increase or reduce the operations of such systems to direct the attention or focus of the PUM or other stakeholder to a specific point. For example, refocusing can include the use of lights for the hard of hearing, alerts or other audible sounds for appliances requiring attention, such as the doors of a refrigerator being open or cooktops not turned off, water running or the like.

In some embodiments, specific focal points, such as doors, windows, pictures, televisions, or other notable points of attention in a SEE can have one or more indicia bound thereto, such as for example QR codes, which in some embodiments may use non visual materials that respond to, for example, infrared or other non-visual wavelengths.

One aspect of the risk simulations can be the measurement of the movements of the PUM, including for example, movement of limbs, so as to ascertain any deviations from the normal patterns of such movements which can, in whole or in part, indicate deterioration of the mobility of that PUM or can indicate an increase in functionality, for example, after a procedure or operation. In some embodiments a PPE can be employed.

In some embodiments, the health condition(s) of the PUM, for example expressed in a HCP for the PUM, may contribute to the contextual risks of that PUM in a specific environment. These contextual risks may be weighted according to the time of day, time periods or locations, such that one or more functions may be employed to calculate the one or more contextual risks of a PUM.

In some embodiments, such contextual risks may contribute to the identification of target risks and consequential calibration, configuration or operation of the one or more sensors, devices or systems present in the SEE for this identification. In this manner, the prioritization or ordering of risks or safety of the PUM may be undertaken. The determination of the most important, timely or relevant risks to a PUM can include the perception of the PUM of those risks. For example, if a PUM has concerns about traversing a set of stairs, these concerns can be included in such risk calculations.

For example, the sensors, devices or systems that are measuring the mobility of the PUM may indicate that the risk for the PUM is (R), whereas the PUM may perceive this risk as (XR), where XR is a multiple of R and XR>R. In this example, the care hubs or care processing systems may communicate to the PUM or other stakeholders, the measured or perceived risk, and may communicate one or more responses to avoid or mitigate such risks. In various embodiments, the PUM may be perceived to be less than the system perceives the risk such that the risk for the PUM is initially identified as (R), whereas the PUM may perceive this risk as (YR), where YR is a multiple of R and YR<R. In various embodiments, when one of the system-determined or PUM-influenced risk determinations (e.g., R, XR, or YR) exceeds a safety threshold, the system may take remediating or investigating actions (e.g., generating an alert, reconfiguring a sensor, etc.), but the system may also take various remediating or investigating actions when a difference between the stem-determined or PUM-influenced risks (e.g., Δ(R, XR), Δ(R, YR)) indicates an anomalous condition, where the PUM has perceived a greater risk that then system has, the PUM has perceived a lower risk that then system has, or the PUM continues with a behavior despite the risks. When the system identifies that the PUM is continuing with a behavior despite the risks identified, the system may perform various additional remediating strategies (e.g., escalating warnings or alerts).

In some embodiments, the combination of SEE generated data sets and PPE data sets can form a set of training data for one or more AI/ML systems. Such data may, for example, be delineated on a temporal basis, where, for example, the state of the PUM and the environment is quiescent. For example, in this case, although the PUM may be undertaking one or more behaviors or patterns, the overall state of the PUM and environment is such that the training data provides the AI/ML systems with an “at rest” or quiescent data representation.

These data can then form, at least in part, part of a simulation of the possible variances to the overall data sets and the subsets thereof, such that the risks to the PUM or other stakeholders may be evaluated by such AI/ML systems. For example, the AI/ML systems may, by changing one or more sensor, device or system data set, predict the potential risk or safety metrics based at least in part on these variances.

This approach can include, for example, behavioral risk assessment, where for example, risk or safety metrics can be impacted by variations in behaviors as measured by the one or more sensors, devices or systems of the SEE. For example, if one or more strain gauges or other haptic sensors detect that the footfall of the PUM is heavier than the quiescent value for that behavior, or one or more audio sensors detects such footfall having a higher or differing audio fingerprint, the one or more care hubs or care processing systems, which can incorporate one or more AI/ML system, digital twin or game theory module, may evaluate these data sets as indicating a decline in the PUM mobility capabilities or an indicator of a health, wellness or safety issue.

In some embodiments, determining the focus of the PUM can be undertaken using the one or more sensors, devices or systems present in a SEE. This determination can include the known patterns and behaviors undertaken by a PUM as part of a regular daily routine as well as data sets of the one or more sensors, device or systems which can indicate the attentions or intentions of the PUM. For example, this determination can include to attention or intention patterns, which for example may indicate the anticipated behaviors or pattern of the PUM, which are then measured by the one or more sensors, devices or systems.

These patterns may, in some embodiments, form the basis of training data for the one or more AI/ML systems, such that the relationships between the intention or attention patterns and the executed behaviors can be determined. In this case, any divergence from the one or more patterns, in anticipation of the executed behavior can be evaluated, and may be represented by variations to the one or more risk or safety metrics, of such patterns or behaviors.

The use of one or more risk or safety simulation systems enables the identification, at least in part, of the personalized risk for a PUM, which may include one or more measured movements, where for example those movements are, in whole or in part, successfully or unsuccessfully completed or such movements form part of a sequence of movements. In this example, each of the movements can have one or more risk or safety metric representing the inherent risk of that movement and one or more other risk metric dimensions representing the degree to which that movement was successfully, or not, completed and in consequence the actual or potential impact of that movement on subsequent movements, patterns or behaviors of the PUM.

The use of one or more PPE calibrated and configured to represent the movements of a PUM can be employed to ascertain the potential movements for that PUM, using for example one or more AI/ML systems that are trained, at least in part, on the actual movements of the PUM, including for example the patterns or behaviors of the PUM as measured by the one or more sensors, devices or systems present in the SEE or employed for the purpose of such monitoring.

The use of such PPE can enable the determination, at least in part, of the potential sequence of movements of a PUM, with the inherent and contextual risk and safety metrics. Such metrics may be based, for example, on mobility frameworks representing the movements of the PUM and the sequences thereof. In this example, each of the mobility frameworks can have an inherent risk metric or a safety metric if applicable, and as such the combination of movements represented by the mobility frameworks can be represented as a sequence of such mobility frameworks that have an aggregated risk. In some embodiments, only when a sequence of mobility frameworks has been executed with the respective risk metrics does a safety metric become calculated.

This calculation can include personalization of a set of risk metrics which in aggregate can have further safety metrics, for example one involving the context of the mobility frameworks and associated risk metrics. For example, if a PUM has a behavior pattern of an early morning run, then the context of that run can have safety impact metrics. For example, if the run is in a local neighborhood, the safety metrics may be low, whereas if that run is in an unknown neighborhood, such as when visiting another city, that safety metric may be higher. In this example, these safety metrics may be communicated to the PUM or other designated stakeholders, including sensors, devices or systems.

The alignment of the one or more risk metrics with the behaviors of the PUM and the determination, at least in part, of the safety metrics for these risks in various contexts can provide such a PUM or other stakeholders with data that can be presented to those stakeholders to inform the stakeholders of such safety concerns. For example, an alert may be created and passed to one or more stakeholders. In some embodiments, communications of the safety metrics may be passed to one or more environment, for example on a campus, the external lighting may be engaged or enhanced as the PUM undertakes a run. These communications can include one or more direct communications of and for the safety of the PUM.

In some embodiments, risks may include both static risks and dynamic risks, both of which may form, in whole or in part, one or more safety metrics. For example, an environment may include an uneven surface, which can be represented by a static risk metric that is higher than a flat even surface. This static risk can be combined with the dynamic risk of the PUM who is traversing such a surface, for example if the PUM has mobility issues, the risk may be increased.

The alignment of the one or more patterns or behaviors of a PUM, based at least in part, on the one or more data sets generated by the one or more sensors, devices or systems present in a SEE and the respective risk metrics for those patterns or behaviors, including those of the environment, locations thereof and the time periods of the patterns or behaviors can represent a personalized risk calculation for one or more PUM. This risk calculation may form, in part or in whole, a risk profile for that PUM as the PUM undertake the various patterns or behaviors. This risk profile, in some embodiments, can form part of or inform the state of the PUM or environment, such as the quiescent state thereof.

In some embodiments, the risk profile may be compared to the data sets generated in real or near real time, by the one or more sensors, devices or systems and the risk metrics thereof to identify any variances from that profile. If any variances are detected, these variances may be communicated to those one or more or to other sensors, devices or systems, including for example, care hubs or care processing to, for example, calibrate, configure or provide operating data to such entities. In some embodiments, such variations may be communicated to one or more AI/ML systems or other predictive systems including for example digital twins, game theory engines or the like in any arrangement.

In some embodiments, each pattern or behavior of a PUM can include, through reference or embedding, one or more risk or safety metrics, for example such metrics may be based, at least in part, on context, including location, time, presence of other stakeholders, sequence of behaviors, events and activities and the like.

These risk or safety metrics may be communicated to one or more risk evaluation systems including one or more risk and outcomes pairings, for example represented by a value key pair or other arrangement. These relationships between risks and outcomes may be, at least in part, derived from one or more knowledge bases containing, for example risks which result in events or actions, one or more risk metrics representing such events or actions or one or more outcomes for such events or actions. For example, this combination may be represented as a tuple, for example in the form of a graph database, where the risk metric N is bound to an event or action X which has, for example a set of potential outcomes (Y1, Y2, Y3 . . . . Yn), where each of these outcomes can be represented by a scalar expressed in terms of one or more impacts on the health, wellness or safety of the PUM.

In some embodiments, a PPE may be employed to identify movements of a PUM, for example as mobility frameworks, where each of these identified mobility frameworks includes one or more risk metrics.

In some embodiments, a PUM may have an intention to undertake a particular behavior, task or action, which can include, for example a sequence of movements or actions, where the PUM initiates that sequence with the intention of completing the sequence, however due to a condition of the PUM or factors influencing these behaviors or actions, the PUM may not be able to complete this sequence. For example, a PUM rises from sitting to traverse to another location in a domicile, for example, the kitchen, and after a few steps feel dizzy, potentially due to blood pressure issues, and needs to return to sitting, either at an origin of the behavior, at a current location, or a nearby chair.

The evaluation of the risks for such an intention can comprise a set of risk variables for each of the movements or actions undertaken by the PUM. For example, the behavior of the PUM may be recognized as a behavior that occurs within a time range from or to a location. For example, a PUM may make tea or coffee at or around 11 am each day. In this case, if the PUM rises at, for example, 11:05 am, then the risk evaluation systems may be configured to assign a risk or safety value to that behavior or intention that is, at least in part, calculated from previous behaviors.

Such a risk evaluation, however, may be varied based on measurements and data from the one or more sensors, devices or systems present in the SEE. For example, if the PUM has a wearable device that can directly or indirectly measure, for example, blood pressure, then the reduction of blood pressure may inform the risk evaluation systems to assign the current activity with a higher risk.

In this example, the risk evaluation systems may communicate such higher risk to one or more sensors, devices or systems such that an alert is communicated to, for example, the PUM or other stakeholders.

If such a situation becomes a more common occurrence, risk evaluation systems may assign a higher risk variable to the initial movements of the behavior, which in some embodiments may generate a message that is communicated to the PUM, through for example, text, audio, visual or haptic means.

In some embodiments, risk categories can be employed to arrange the behaviors of one or more PUM. For example, this can include categories based on mobility frameworks, such as standing, walking, sitting, and the like or behaviors such as making lunch, dinner, coffee and the like, exercise, sleeping or resting and the like. Each of these categories can have a set of outcomes or impacts that represent the possible safety, health or wellness outcomes or impacts for one or more PUM. These categorized behaviors can have one or more risk or safety metrics represented, for example as characteristics of those behaviors. Each of the categories or the behaviors thereof can have relationships with the potential outcomes or impacts of the one or more potential safety, wellness or health outcomes or impacts that can occur. For example, in the exercise category, there may be multiple outcomes, such as twisted ankle, hyperextended elbow, pulled neck or back muscle and the like, which can be the result of such exercise. In each of these cases, which in turn may be represented by a classification, including for example an ontology, taxonomy or other organizational arrangement, there can be a risk or safety metric which can represent the impact of such safety, wellness or health outcome.

Such a classification system can include the probability of occurrence of such impacts, which in some embodiments may be calculated from, for example, a PPE of the PUM or one or more AI/ML systems in any arrangement. This probability metric can, in some embodiments, inform one or more systems, including for example safety enabled devices, care hubs, PERS devices or other care processing systems. These devices or systems may then generate one or more communication to the PUM or other stakeholders, for example, in the form of a message or alert, indicating the risk of the current or likely activity. These messages may be communicated, for example through text, audio, visual, or haptic or other methods.

In some embodiments, the combinations of categories, behaviors or patterns thereof, the safety or risk metrics of those categories, the potential outcome and impacts of such behaviors or patterns and the safety and risk metrics in combination with the one or more probability or predicted impacts or outcomes may be represented, for example as a multi-dimensional matrix or other multi-dimensional data set.

In some embodiments, these categories and the safety and risk metrics may be represented in the form of risk or safety vectors, which can inform one or more safety enabled device, PERS, care hub or care processing system, such that the PUM or other stakeholders can be presented with a simplified projection, in terms of the risks to the safety, wellness and health based on the current behaviors and the potential outcomes of such behaviors.

In some embodiments, such metrics can have one or more sets of weightings that are, at least in part, determined from the behaviors of one or more PUMs, including for example a corpus of PUMs with similar health and wellness conditions, locations, physical or other attributes or the like.

In some embodiments, risk or safety metrics can have or include weightings, multipliers or other functions that operate on a set of default risk or safety metrics, including those represented by a constant. For example, if a PUM regularly initiates and completes a pattern or behaviors, the variable function of the risk or safety metrics for that pattern or behavior may be varied to represent these successful completions. This variable may be represented, for example, as a vector which indicates the state of the PUM or other stakeholders in relation to this pattern or behavior, such that the underlying risk of the pattern or behavior is recognized as such, although that PUM undertaking that pattern or behavior can have further representations. In this manner, should the PUM improve, for example, after a medical procedure, physical therapy, medication or other variation to the health or wellbeing of the PUM, this improvement can be represented by such a vector. In another example, if the PUM has an increasing difficulty in completing such patterns or behaviors, then this variable can represent such a decline in capability.

The use of these vectors by one or more AI/ML systems can provide insights into the likely trajectory of a PUM in relation to these patterns or behaviors. These insights can be used to calibrate, configure or operate the one or more sensors, devices or systems to identify variations in these vectors at the earliest possible time to inform, for example, one or more care hubs or care processing systems or one or more other systems including response systems, IAS and the like. This approach can provide a set of data that is correlated to the variations of the PUM patterns or behaviors that have one or more relationships to the underlying risks of the patterns or behaviors, including inherent, contextual, locational or temporal, to identity those variations that are indicative of improvements or declines in a PUM's health, wellness or safety and the capability of the PUM in this regard at the earliest opportunity.

One aspect of this approach may be determining, at least in part, the confidence with which a PUM undertakes a pattern or behavior. For example, if the PUM has declining eyesight capabilities, which tend to be incremental, then confidence metrics, represented at least in part by such vectors, can be informing as to the need for that PUM to address these eyesight capabilities. This approach can also apply to mobility or other physical attributes of the PUM as well as indications as to the mental state of the PUM. This adaptation can include actions by the PUM, for example, obtaining new glasses, actions by a carer or other stakeholder or variations in the calibration or configuration of the SEE or the one or more sensors, devices or systems therein, which can include those present, such as mobile devices and the like.

The alignment of the one or more risk or safety metrics with patterns or behaviors can, in some embodiments, be represented by measured or calculated variables or derivatives or secondary variables, such that for example, the locational risks are expressed as a value (N), and the presence of a PUM undertaking behavior (A), can have a set of variables for that behavior which can differ from a set of variables if the PUM is undertaking behavior (B).

The use of the one or more AI/ML systems can predict or monitor the trends of the patterns or behaviors of the PUM, based at least in part, on these vectors or other weighting or variables that have one or more relationships with the underlying risk or safety metrics.

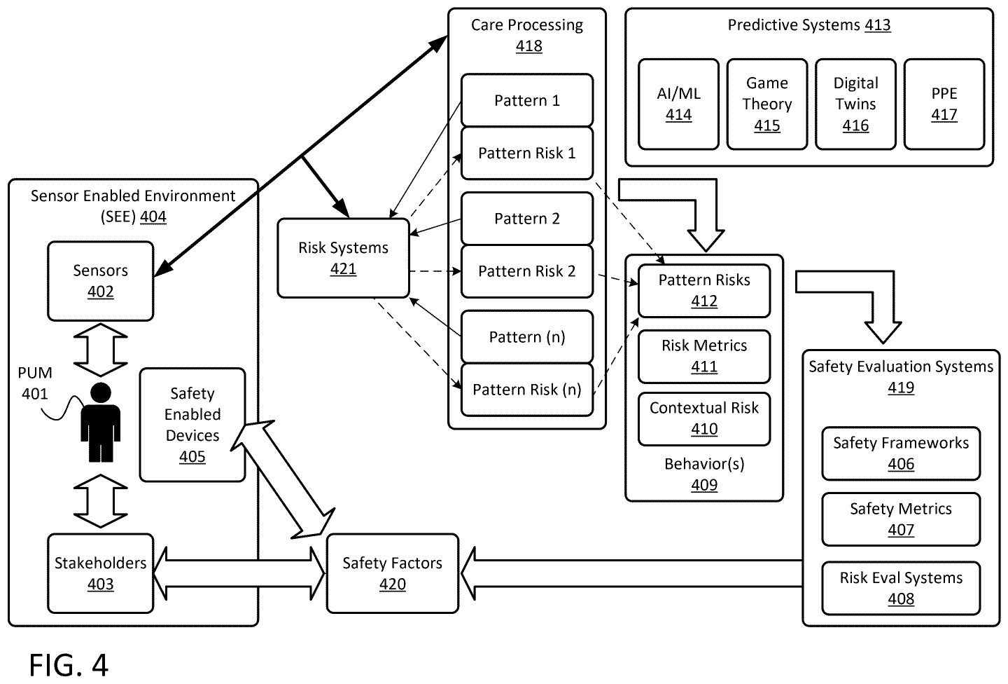

FIG. 4 illustrates an example embodiment of a safety evaluation system where a SEE 404 includes a PUM 401, one or more sensors, devices or systems 402, one or more stakeholders 403 and one or more safety enabled devices 405. The data generated by the sensors 402 can be evaluated by one or more care systems 418, including for example, care hubs, where one or more patterns, for example pattern 1, 2 . . . (n) are determined and using one or more risk systems 421, the one or more risk metrics are determined for such patterns, for example pattern risk 1, 2 . . . (n). These pattern risks 412 are aggregated and combined with contextual risks 410 to generate one or more set of risk metrics 411 for one or more behaviors 409. These behaviors 409 and the risk metrics 411 thereof can be communicated to one or more safety evaluation systems 419, which can include safety frameworks 406 and risk evaluation systems 408, which can in combination generate one or more safety metrics 407, which in aggregate can be represented by one or more safety factors 420. These safety factors can be communicated to one or more safety enabled devices 405 or to one or more stakeholders 403, including the PUM 401, in human interpretable form.

In some embodiments, the impact of an event, action or response which is the outcome of one or more behavior or pattern involving a PUM can be represented in the form of an impact metric. For example, these metrics can include one or more scalar representing, at least in part, the actual or potential health, wellness or safety impact on one or more PUM or other stakeholders. For example, these impact metrics can be used by one or more IAS to, at least in part, evaluate a response to a PUM behavior or pattern and may be used to adjust that response.

In some embodiments, outcomes of activities involving a PUM can have known relationships with one or more impacts of those outcomes. For example, if a PUM stubs a toe on a piece of furniture, this event can have a number of correlated impacts, where for example, if the PUM is on blood thinners, the impact may be more significant than if not. These relationships can, in some embodiments, be used by an IAS.

In some embodiments, one or more organizations of outcomes, responses or impacts may be stored in one or more repository, for example a knowledge base. The knowledgebase can include the classification and organization of these outcomes, responses and impacts based on, at least in part, severity of impact on a PUM or other stakeholder, temporal immediacy, response capabilities, or other factors that can have an impact on the health, wellness or safety of a PUM.

One aspect of the impact analysis, for example using an IAS, is the use of predictions to ascertain, at least in part, the “what-if” for one or more outcomes, the responses thereto and the impacts thereof. This analysis can lead to significantly complicated branching of the potentialities for these considerations, in that with three prime entities, each having multiple possibilities, the predictions can soon become unmanageable.

The use of typical embodiments, such as graph databases, decision trees, structured databases, ontologies, taxonomies and the like can soon become overwhelmed, where the possible outcomes and the probabilities, let alone the potential impacts, are of such a magnitude as to be impractical. This overwhelming effect is particularly noticeable in the case where a timely impact analysis for one or more responses is required. For example, the computing resources needed to perform an N-complex tasks, which branches into additional N-complex tasks may be too great to provision on various hardware used by the system for maintaining the safety of the PUM, while providing timely results to avoid hazardous situations.

The use of one or more classification schemes, including ontologies or taxonomies, can be employed to create various groupings of outcomes, responses and impacts, based at least in part on similarities of the attributes of those entities, including for example risk or safety metrics, these organizations can be used to inform other systems, though for example, limited depth of branching that can make such systems practical.

To address this issue and provide actionable results in an appropriate timeframe, one or more AI/ML systems may be employed, which can include LLMs, deep learning, sparse data systems, neural networks, RAGs, LCMs and other AI/ML systems. In some embodiments, game theory engines may be employed to represent simplifications of the potential relationships between outcomes, responses and impacts, where, for example, one or more corpuses of such relationships provide game frameworks that indicate the appropriate strategies and payoffs. The combination of AI/ML systems, including for example, specialized response or impact systems, for example, those incorporating Recurrent Neural networks (RNN's) and the like and one or more game theory engine with organizations of behaviors, patterns, events, actions or other measured occurrences, outcomes of those occurrences, including predicted or actual, and one or more candidate responses and the one or more potential or actual impacts of such responses, can be formulated to represent a timely, effective and efficient response to the occurrence. The use of this approach, where, for example, certain occurrences, including those pertaining to the health, wellness or safety of a PUM, such as behaviors, patterns, events, actions and the like, such as tripping on carpet, falling, breathing difficulties and the like, can be correlated with one or more responses and the potential impacts of the occurrence of the responses, an IAS may be used to configure such responses or the one or more sensors, devices or systems of the SEE for the most immediate and effective response.

In some embodiments, this approach can include AI/ML models that have been trained in conjunction with game theory systems, so that a set of response candidates may be made available with utmost timeliness. This approach can include sets of models, simplifications and responses that can, for example, be configured and hosted on devices with limited resources, such as PERS devices, smart phones, smart watches, fitness trackers and the like.

In some embodiments, one or more game theory modules may operate in conjunction with, for example an LLM or LCM, to generate one or more potential impact analysis, which can represent a history based perspective, which can then be compared to an LLM or LCM generated outcome and any variations used, for example, as feedback to either or both systems.

In some embodiments, one or more Large Language Models (LLM), which combine embedding, the process of transforming input data, such as words or tokens, for example data sets from the one or more sensors, devices or systems or declarations from the one or more PUMs or stakeholders, into a dense vector representation, and attention mechanisms, a technique that focuses on specific parts of an input data sequence when generating output, allowing the model to selectively weigh the importance of different input data elements, rather than treating them equally, may be employed.

As will be appreciated, in an LCM, a “concept” is an atomic idea, which may be represented in various formats. In an example using text, the concept of “falling” may be described in the first sentence of: “the PUM descended from a standing state to a lying down state at a speed and acceleration consistent with gravitational pull” or the second sentence of: “a force of impact on a floor sensor consistent with an object of 50-75 kilograms accelerating at 9.8 meters per second per second over a distance of 1-2 meters has been detected”. The present disclosure contemplates that various states or behaviors may be used as concepts within the LCM (rather than or in addition to a text representation). Additionally, an LCM can use various sensor archetypes that are able to represent the same concept (e.g., detection models that are audio based, video/image based, haptic based, etc. and combinations thereof) to provide an initial level of monitoring based on the available or permitted (e.g., according to a privacy profile) and confirm that the same concepts are detected via another monitoring paradigm to reduce or eliminate hallucinations in the models by confirming that the various events are coherent (e.g., congruent) with one another or in series. In various embodiments, the LCM is configured to check for global coherence within a window of ongoing concepts (e.g., determining whether concepts when viewed as a collective series in the window are logically appropriate with one another).

This combination of techniques allows LLMs and LCMs to process data with complex relationships, interpret those relationships based on context, and predict possible outcomes. This ability makes LLMs effective as text and human language processing tools. These capabilities can also be applied to other data representing complex patterns, for example data sets, patterns or behaviors generated by the one or more sensors, devices or systems present in a SEE, including movements that are part of a person's activity, which can enable predicting the person's intention or the outcome, including end state, from a sequence of movements, including body part movements, such as mobility frameworks, whole body movements, such as patterns or behaviors, within an environment, eye focus changes or other indicators of attention and intention.

As will be appreciated, in an LLM, a “token” is a division of data used for analysis and prediction. In an example using text, a series of tokens of individual words of “The PUM stepped on a slippery surface and . . . ” may predicted the next token as “fell” to identify the outcome of a set of states. The present disclosure contemplates that various states or behaviors may be used as tokens in the LLM (rather than or in addition to text representation of the text). In various embodiments, the LLM is configured to check for local coherence for ongoing tokens (e.g., determining whether the next token in a series of tokens is logically appropriate).

As will be appreciated, in an LCM, a “concept” is an atomic idea, which may be represented in various formats. In an example using text, the concept of “falling” may be described in the first sentence of: “the PUM descended from a standing state to a lying down state at a speed and acceleration consistent with gravitational pull” or the second sentence of: “a force of impact on a floor sensor consistent with an object of 50-75 kilograms accelerating at 9.8 meters per second per second over a distance of 1-2 meters has been detected”. The present disclosure contemplates that various states or behaviors may be used as concepts within the LCM (rather than or in addition to text representation of the text). Additionally, an LCM can use various sensor archetypes that are able to represent the same concept (e.g., detection models that are audio based, video/image based, haptic based, etc. and combinations thereof) to provide an initial level of monitoring based on the available or permitted (e.g., according to a privacy profile) and confirm that the same concepts are detected via another monitoring paradigm to reduce or eliminate hallucinations in the models by confirming that the various events are coherent (e.g., congruent) with one another or in series. In various embodiments, the LCM is configured to check for global coherence within a window of ongoing concepts (e.g., determining whether concepts when viewed as a collective series in the window are logically appropriate with one another).

In some embodiments, embedding maps each data input, or token, to a unique vector, typically using a lookup table or a neural network-based technique, or a combination of both approaches, to map each token to a unique vector. The vector values represent what the “meaning” of the data token is within the context of the system. For example, the vector values for text tokens can represent the multi-dimensional meaning of the token or word in the human language that the word belongs to. A mapping between, for example, a person's movements and the most common implications, intentions, outcomes, and the like, of those movements can be constructed by using data analytics or machine learning techniques.

In some embodiments an LLM-based architecture that incorporates a custom-designed embedding mechanism to transform raw data (e.g., sensor, device or system data and measurements, including image or other frames) into dense vector representations. These embeddings can then be processed through an LLM attention mechanism that selectively attends to relevant features and relationships within the input data, allowing for accurate prediction of human movement intentions, outcomes, and risks of potential neutral, positive or negative health, wellness or safety events. This method enables the prediction of risks associated with complex user behavior patterns, such as falls, accurate prediction of falls in elderly or disabled individuals to prevent injuries; cardiovascular events, early warning system for predicting cardiovascular or similar events, such as heart attacks or strokes; injury risk assessment, real-time evaluation of the likelihood of injury based on user behavior or environmental factors.

This method also offers several advantages over existing techniques, including, improved accuracy, robustness to noisy or incomplete data, improving overall system reliability, and flexibility, enabling adaptation to various input modalities and applications.

In some embodiments, the result of the one or more systems employed within a SEE may be one or more calibrations, configurations, commands, specifications or instructions which may be represented, for example, by one or more tokens. These results can be passed from the systems generating such to one or more action execution systems, which can provide the one or more interfaces to the sensors, devices or systems that can interpret or act upon these commands, specifications or instructions. For example, these sensors, devices or systems can include home automation systems, IoT devices, SEE present sensors, devices or systems, third party systems, such as emergency systems and the like.

In some embodiments, one or more action execution systems can communicate with, for example, using tokens, one or more sensors, devices or systems to calibrate, configure or command these one or more sensors, devices or systems to undertake one or more actions based on their capabilities.

In some embodiments, the combination of the data sets generated by the one or more sensors, devices or systems of the SEE in combination with the data generated by a simulation system, for example one or more digital twin, can create a data set that may include one or more risk and safety metrics for these actual and simulated data sets to, at least in part generate, using one or more systems, including risk management systems, care hubs or care processing a set of responses to the actual or simulated data sets representing the situation of the PUM, both in real time and in a time progression.

Each of these response sets can have one or more outputs, for example communications with a PUM or stakeholders, calibration, configuration and/or operation of one or more sensors, devices or systems present in a SEE, communications, including alerts to third party services or the like.

In some embodiments, such responses may be represented by a set of vectors representing the sequence of events that unfold based on the initial response conditions. These vectors can include the relationships and interactions between the one or more entities, including for example the one or more sensors, devices or systems present in a SEE, the one or more PUM or other stakeholders, one or more systems, such as care hubs, care processing systems, one or mor risk or safety systems or the like.

In some embodiments, there may be one or more safety enabled devices, where a device which is worn or carried by a PUM or is embedded in an environment is configured to provide the PUM with safety communications. For example, the safety enabled devices can include the use of text, visual, haptic, audio, electrical or other forms of communication with the PUM and can include one or more representations of these safety communications, for example as discrete quantized communications, such as “Safe/Unsafe” or graduated safety communications, for example expressed as a color or graduated visual representation.

In some embodiments, devices typically worn or carried by a PUM may be enabled through, for example, an application that is hosted or resident, either through reference or embedding, on that device, such as smart phones, PERS devices, fitness or other trackers, other wrist worn devices or pendants or the like.

In some embodiments, a PUM may have a personalized risk device, which may include one or more communication capabilities including, for example, visual, audio or haptic communication means that can inform the PUM of the risks of a particular behavior. Such a device can include communications capabilities such that the PUM can indicate to the device the intended behaviors of the PUM, including sets or sequences thereof, and the device can provide risk metrics or other communications for these intended actions.

In some embodiments, a safety enabled device may alert the PUM or another stakeholder of the potential safety considerations of one or more behaviors or activities. Safety considerations can include those behaviors or activities that correspond to the time of day, location, previous behaviors, interactions with other stakeholders and the like.

For example, if a PUM exhibits a behavior at a particular location or time of day, the safety enabled device may communicate with the PUM, so as to alert the PUM to any safety concerns that the one or more sensors, devices or system present in the SEE and the one or more risk systems thereof, may be aware of. These risks can include those risks that, for example, using a PPE, Digital Twins, AI/ML modules or other predictive systems, that have a corresponding safety risk.

In some embodiments, these safety alerts may be, in whole or in part, controlled by the PUM, in that the PUM may configure the safety enabled device to provide such alerts only in certain circumstances and may select one or more methods of communication, including for example, by text, audio, video or haptic methods.

In some embodiments, a safety enabled device can incorporate biometric or physiological sensors, including, for example, sensors for measurements of skin electrical characteristics, stress indicators, direct and indirect eyesight and attention and focus measurements, and the like.

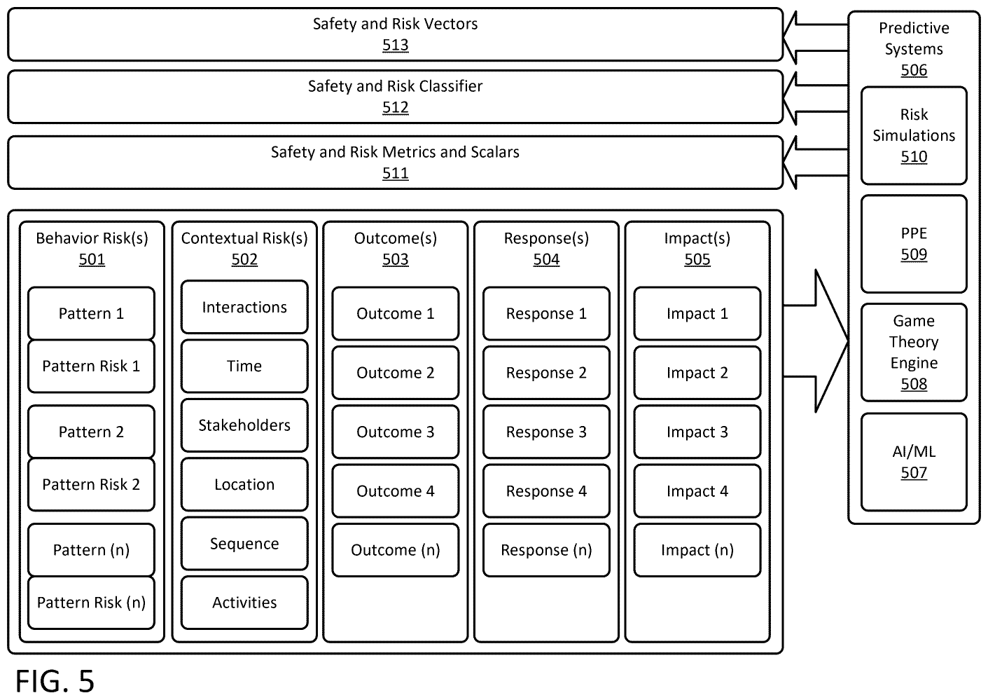

FIG. 5 illustrates an example embodiment where the patterns and risk metrics 405 can be combined with contextual risks 502 and, using one or more risk or safety system, one or more outcomes 503 can be generated, which can be communicated to one or more response systems to generate one or more responses 504, which can be communicated to one or more impact analysis systems 505, all of which can, in part or in whole, be communicated to one or more predictive systems, which can include, in some embodiments, one or more AI/ML systems 507, that, for example, include one or more LLM, LCM, one or more game theory engine 508, one or more PPE 509 and one or more risk simulations systems 510, all of which may operate and interact in any arrangement so as to, in some embodiments, generate one or more safety and risk metrics and scalars 511, which may be evaluated by or operated on, by one or more safety and risk classifiers 512 which can contribute to or inform one or more safety and risk vectors 513.

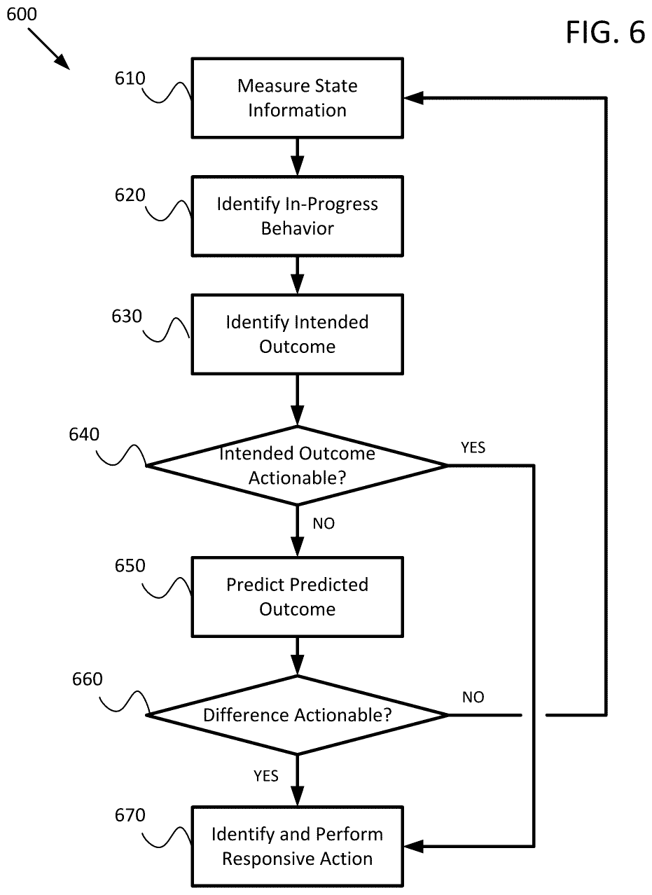

FIG. 6 is a flowchart of an example method 600 for automated anomaly detection and response generation in a sensor-enabled environment, according to embodiments of the present disclosure. Method 600 begins at block 610, where a computing system that is part of or used in association with a SEE in which a person under monitoring (PUM) is monitored measures state information of the SEE and the PUM via a plurality of sensors disposed in SEE. The SEE includes a plurality of sensors, which may include sensors worn, carried, or implanted in the PUM and sensors, and sensors disposed in the SEE that are not worn, carried, or implanted in the PUM, all of which may provide various data at various rates, frequencies, granularities, encodings, formats, and encryptions, which one or more artificial intelligence or machine learning (AI/ML) models use to monitor the health, wellness or safety of the PUM and other parties in the SEE.

At block 620, the system, using the state information, identifies an in-progress behavior affecting the PUM in the SEE. In various embodiments, the in-progress behavior may be performed by one or more entities, which may include the PUM or other stakeholders for care of the PUM. For example, the behavior may be performed, at least in part, by the PUM, or by a stakeholder for care of the PUM other than the PUM, wherein the other stakeholder is selected from the group consisting of: a caregiver of the PUM; a friend of the PUM; a neighbor of the PUM; a family member of the PUM; an insurance provider for the PUM; a medical professional; an emergency responder, or other identified stakeholder.

In various embodiments, an AI/ML system uses the state information and optionally various previously measured state information to identify the in-progress behavior. For example, a first location of the PUM within the SEE and first relative locations of the limbs of the PUM measured at a first time and a second location of the PUM within the SEE and second relative locations of the limbs of the PUM measured at a second time can be used as state information to identify that the PUM is standing up from a sitting position from the first time to the second time as an in-progress action that the PUM is currently performing.

In some embodiments, the in-progress behavior includes providing a medication, dietary supplement, food or beverage to the PUM and the actionable risk level for health, wellness or safety of the PUM is identified (per block 640 or block 660) based on an effect selected from the group consisting of: an ability of the PUM to feed or administer to themselves the medication, dietary supplement, food or beverage; a swallowing difficulty of the medication, dietary supplement, food or beverage; an interaction or metabolic rate effect between the medication to the PUM and the dietary supplement, food or beverage; an interaction or metabolic rate effect between the dietary supplement taken by the PUM and the medication, food or beverage; an allergy, intolerance or sensitivity of the PUM to the medication, dietary supplement, food or beverage; a temperature of the medication, dietary supplement, food or beverage; a blood sugar deviation; and an adherence of the medication, dietary supplement, food or beverage to a medical, religious or conscientious dietary plan for the PUM (e.g., Clear Liquids Only, Kosher, Halal, Vegetarian, Vegan, etc.).

In some embodiments, the in-progress behavior includes movement within the SEE and the actionable risk level for health, wellness or safety of the PUM is identified (per block 640 or block 660) based on a status selected from the group consisting of: a state of an appliance in the SEE; a light level in the SEE; a time of day; a temperature in the SEE; a presence of a person or animal in the SEE; a medication taken by the PUM; a food or beverage consumed by the PUM; and a presence of liquid on a floor of the SEE.

In some embodiments, the in-progress behavior includes a conversation that includes the PUM and the actionable risk level for health, wellness or safety of the PUM is identified (per block 640 or block 660) based on sharing private information with another party to the conversation or present in the SEE that is not authorized to receive the private information. For example, to prevent a phone scammer from receiving banking, financial, medical, or other personally identifiable information from the PUM or another stakeholder engaging in a phone call.

In some embodiments, the in-progress behavior includes a repeated action not necessary to be performed more than once for achieving the intended outcome, and the actionable risk level for health, wellness or safety of the PUM is identified (per block 640 or block 660) with a dementia event. For example, if the PUM is identified as engaging in a behavior to move to the kitchen, but walks back to the sofa, then back to the kitchen, then back to the sofa, then back to the kitchen, etc., the repeated behaviors are not conducive to achieving an intended outcome of getting a meal or beverage, and may be indicative of a “wandering” type dementia event.

At block 630, the system identifies an intended outcome of the in-progress behavior. Various behaviors may have one or several intended outcomes that the system can identify as intended by the entities performing the actions that make up the behavior. For example, a PUM engaged in a behavior of “going to the kitchen”, which may be associated with intended outcome of “the dishes in the sink are cleaned”, “lunch is prepared”, or “a drink is prepared”. For example, a carer engaging in the behavior of “making a sandwich” may have an intended outcome of “feeding the PUM lunch”.

In some embodiments, the intended outcome is based on historical data associated with the behavior identified as being in progress. For example, when the PUM is historically noted as moving from the living room to the kitchen around noon to make lunch, and the PUM is observed as performing an in-progress behavior of moving from the living room to the kitchen around noon, the historical intent of “make lunch” may be assigned to the in-progress behavior.

In some embodiments, the intended outcome is based on an utterance or communication from the stakeholder engaged in the behavior. For example, a PUM may state “I am making lunch”, which microphones in the SEE collect and process to identify the intent of the PUM. In another example, a carer may be called into the SEE by the PUM to perform a task, and the message used to call the carer is parsed to identify the intent.

In some embodiments, the states and behaviors identified in the SEE are used as tokens by an LCM/LLM AI/ML system, which identifies the intended outcome as a next state or behavior in a series of states and behaviors using the data available to the PUM or stakeholder, which may be different from the total data available to the SEE. Accordingly, the LCM/LLM AI/ML system may make use of one or more digital twins of the PUM that have limited amount of information about the SEE provided to the PUM to identify what the intent of the PUM may be given the information available to the PUM. For example, if the system knows that a bathtub has overflowed in a first bathroom, and observes that the PUM is engaged in the behavior of walking to that bathroom, one or more of the digital twins may be denied the information that the bathtub has overflowed (despite the system having this information). Accordingly, using the information available to the PUM, the system may determine the intent of the PUM when walking to the first bathroom to be “using the bathroom” rather than “stopping the water in the bathtub”, even if the intent of the PUM would be “stopping the water in the bathtub” had the PUM known of the overflow.

At block 640, the system determines whether the intended outcome represents an actionable risk level for the health, wellness or safety of the PUM. For example, some behaviors or activities may inherently be risky to the health, wellness or safety of the PUM, such as when the PUM attempts to smoke a cigarette, change a lightbulb in a ceiling-mounted fixture, move a heavy object, etc. Accordingly, when the system identifies that the performance of the behavior as intended represents an actionable risk level to the PUM, method 600 proceed to block 670, and optionally continues to block 650. When the system determines that the intended outcome is not actionable (e.g., quiescent), method 600 proceeds to block 650.

In various embodiments, the quiescent outcomes represent the outcomes of behaviors that do not cause the system to generate an alert or other follow-up action to a stakeholder, whereas actionable outcomes cause the system to generate an alerted of rother follow-up action. Depending on the HCP and supporting data for the PUM, two stakeholders may engage in the exact same behavior with the same intended (or predicted) outcomes, where the system may identify the outcome for a first stakeholders as actionable and for the second stakeholders as quiescent. For example, a first PUM with no indications for visual or mobility impairments may be identified as attempting to walk through a room where water is present on the floor, and is judged to not be at sufficient risk of falling that the behavior is identified with a quiescent outcome (e.g., not worth sending an alert over). In contrast, a second PUM with indications of visual or mobility impairments who may not see or be able to retain balance in light of a fall hazard is judged to be at sufficient risk of falling that the behavior of attempting to walk through a room where water is present on the floor is identified with an actionable outcome.

At block 650, the system predicts, using the state information and the in-progress behavior, a predicted outcome of the in-progress behavior. Some of the outcomes may be intended or unintended consequences to the outcome of the in-progress behavior. The system may use a PPE or various digital twins of the PUM or other entities in the SEE to predict outcomes from the behaviors. These predicted outcomes may include subsequent outcomes to the intended outcomes of the in-progress behavior, alternative outcomes (compared to the intended outcome) from the in-progress behavior, or subsequent outcomes to the alternative outcomes (e.g., subsequent-alternative outcomes).

For example, when a PUM is engaged in the behavior of “going to the kitchen” with an intended outcome of “cleaning the dishes”, one subsequent outcome to performing “preparing a meal” may be a follow-up behavior performed after the in-progress behavior of “leave the kitchen” with a subsequent outcome of “the PUM falls due to water on the floor”, one alternative outcome may be that “the PUM prepares a meal”, and one subsequent-alternative outcome to preparing the meal may be that “the PUM leaves the stove unattended”.

For example, when a carer is engaged the behavior of “making a meal for the PUM” with an intended outcome of “feeding the PUM”, one subsequent outcome to “feeding the PUM” may be that “the PUM chokes on the food”, one alternative outcome may be that “the carer eats the meal instead”, and one subsequent-alternative outcome to the carer eating the meal may be that “the carer falsely reports that the PUM was fed”.

In some embodiments, the states and behaviors are used as tokens by an LCM/LLM AI/ML system, which identifies the predicted outcome as a next state or behavior in a series of states and behaviors. The system may use data that are not available to the stakeholders, or information that has been forgotten by the stakeholders engaged in the behaviors to identify potentially unintended consequences to the behaviors. For example, a PUM may not know that there is water present on the floor in another room, but the system identifies the presence of the water to identify that an outcome of entering that room may include an elevated risk of the PUM slipping and falling. For example, a PUM may have received a new prescription that a carer is unaware of and that the PUM has forgotten about, and the system may identify the side effects as including vertigo, which may elevate the risk of the PUM falling when performing particular behaviors.

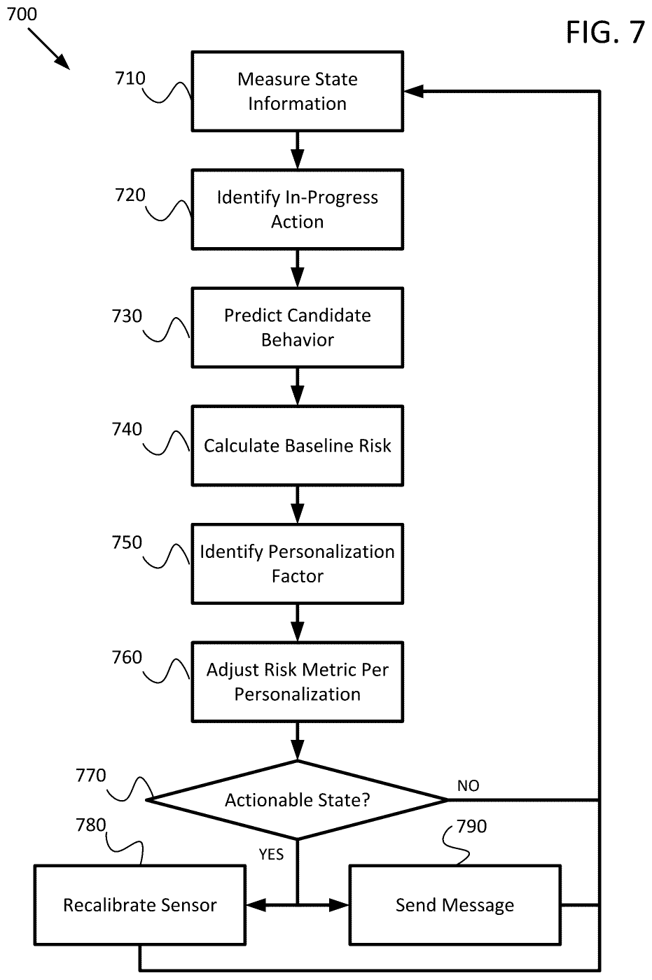

As will be appreciated, some of the predicted outcomes may indicate an actionable risk level to the health, wellness or safety of the PUM, while other predicted outcomes may indicate innocuous alternatives. However, the likelihood of a particular predicted outcome occurring, may be affected by various factors, such that the risk to the PUM is determined based on a balance of the severity of the outcome and a probability of the outcome occurring. The AI/ML system may provide various weights to the likelihoods and severities of the outcomes, and may develop baseline risk profiles for the outcomes of various behaviors that may be personalized to the scenario in which the PUM is engaged by location, time, and attitude of the PUM to the task and outcomes thereof. One example method of risk personalization is discussed in relation to method 700 with respect to FIG. 7.

At block 660, the system identifies whether a difference between the intended outcome and a predicted outcome represents an actionable risk to the health, wellbeing or safety of the PUM. Because not every difference warrants alerting the PUM or other stakeholder for care of the PUM, or taking another action, in responsive actions may be taken selectively.

For example, when the intended outcome is identified as “get a drink from the kitchen” and the predicted outcomes include “get a snack from the kitchen” as an alternative, the difference in risk to the PUM would be expected to be negligible to the PUM in most situations. However, if the predicted outcomes include an unintended consequence, such as a subsequent outcome of “falling while navigating kitchen” due to a sensor-identified puddle on the kitchen floor, the risk may be classified as actionable.

In various embodiments, the difference in risk levels may be assessed against various thresholds, Boolean checks, consensus models (e.g., among a plurality of digital twins), and combinations thereof to determine when a difference is actionable versus quiescent.

In various embodiments, identifying whether the difference between the intended outcome and the predicted outcome represents an actionable risk level for health, wellness or safety of the PUM is determined, at least in part, by analyzing at least one of: data from a health care profile (HCP) of the PUM; historical behavior patterns of the PUM; and contextual data of the SEE for a time or location where the in-progress behavior is being performed.

In various embodiments, identifying whether the difference between the intended outcome and the predicted outcome represents an actionable risk level is identified with a mental state event associated with an emotional response by the PUM, wherein the emotional response includes the PUM uttering at least one word or phrase, wherein the emotional response is selected from the group consisting of: yelling; crying; screaming; hyperventilating; tachycardia; a spike or a drop in blood pressure of the PUM; a self-soothing behavior; and a pain response.

In response to determining that the difference represents an actionable risk to the health, wellbeing or safety of the PUM, method 600 proceeds to block 670; otherwise, method 600 may return to block 610 to continue monitoring the PUM.

At block 670, the system identifies and performs a responsive action to the in-progress behavior to reduce the actionable risk level to the PUM. Depending on the type of the in-progress behavior and whether the in-progress behavior itself or a difference from an intended outcome and a predicted outcome resulted in the system performing block 670, the system may select from various responsive actions to learn more about the SEE or the PUM, alert the PUM or another stakeholder, or affect a system within the SEE to mitigate or avoid risk to the PUM.

In some embodiments, performing the responsive action includes transmitting a message to the PUM or at least one other stakeholder for care of the PUM that identifies the predicted outcome of the in-progress behavior. For example, the PUM may be warned of a slip hazard before entering a room so that the PUM may alter a gait, ask for assistance, or choose an alternative action to reduce the risk.

In some embodiments, performing the responsive action includes transmitting a message to the PUM or at least one other stakeholder for care of the PUM that identifies an alternative behavior associated with the intended outcome with a lower risk assessment than the in-progress behavior. For example, when a first bathroom is identified as having water on the floor, the system may recommend that a second bathroom be use to achieve the same result of a behavior of “going to the bathroom”.

In some embodiments, performing the responsive action includes reconfiguring at least one sensor of the plurality of sensors by sending a configuration command for the at least one sensor of the plurality of sensors from the group consisting of: activating the at least one sensor of the plurality of sensors; deactivating the at least one sensor of the plurality of sensors; and increasing a granularity of data collected by the at least one sensor of the plurality of sensors; decreasing the granularity of data collected by the at least one sensor of the plurality of sensors; increasing a reporting rate of the at least one sensor of the plurality of sensors; decreasing the reporting rate of the at least one sensor of the plurality of sensors; and changing an optical focus of the at least one sensor of the plurality of sensors.

In some embodiments, performing the responsive action includes: transmitting a control signal to a device disposed in the SEE that performs a safety action selected from the group consisting of: shutting off an appliance; engaging or disengaging a lock on a door or window; opening or closing a door or window; turning on or off a light; and terminating a telephone call.

FIG. 7 is a flowchart of an example method 700 for automated anomaly detection and response generation in a sensor-enabled environment, according to embodiments of the present disclosure. Method 700 begins at block 710, where a computing system that is part of or used in association with a SEE in which a person under monitoring (PUM) is monitored measures state information of the SEE and the PUM via a plurality of sensors disposed in SEE. The SEE includes a plurality of sensors, which may include sensors worn, carried, or implanted in the PUM and sensors, and sensors disposed in the SEE that are not worn, carried, or implanted in the PUM, all of which may provide various data at various rates, frequencies, granularities, encodings, formats, and encryptions, which one or more artificial intelligence or machine learning (AI/ML) models use to monitor the health, wellness or safety of the PUM and other parties in the SEE.

At block 720, the system, using the state information, identifies an in-progress action for the PUM in the SEE. In various embodiments, an AI/ML system uses the state information and (optionally) various previously measured state information to identify the in-progress action that the PUM is currently performing. For example, a first location of the PUM within the SEE and first relative locations of the limbs of the PUM measured at a first time and a second location of the PUM within the SEE and second relative locations of the limbs of the PUM measured at a second time can be used as state information to identify that the PUM is standing up from a sitting position from the first time to the second time as an in-progress behavior that the PUM is currently performing.

At block 730, the system predicts, using the state information and in-progress action, a candidate behavior for the PUM to perform within the SEE that includes the in-progress action and a candidate subsequent action to be performed after the in-progress action as part of the candidate behavior. In various embodiments, an AI/ML system uses the state information and the current in-progress action (identified per block 720) to identify the in-progress action that the PUM is currently performing.

In various embodiments, the AI/ML system can use previously identified in-progress actions, which may now be completed, to inform the prediction of a candidate behavior that the PUM in engaged in.

In various embodiments, the AI/ML system can identify several candidate behaviors that the PUM is potentially engaged in, and model the different behaviors via different digital twins of the PUM and SEE. In various embodiments, the system may model the N most-likely candidate behaviors in parallel, but may prune simulation when a candidate behavior is no longer possible, or falls below a threshold probability of being performed by the PUM. For example, where N=3, the system may identify three candidate behaviors of the PUM “going to the bathroom”, “going to prepare a meal”, and “going to the garden” as the most likely to be performed by the PUM based on an action of “rising from the sofa”, and the system therefore model three instances of the PUM and SEE; one instance for each of the three identified tasks.

Other candidate behaviors (e.g., the 4^{th} most-likely behavior when N=3) may also be identified, but may not initially be modeled. Additionally, as additional actions and states are identified in the SEE, the system may cease modeling and predicting based on behaviors that are no longer likely, or may begin modeling and predicting based on behaviors becoming more likely. For example, a candidate behavior of “doing laundry” may be identified as the 4^{th} most-likely candidate behavior based on the PUM performing an action of “rising from the sofa”, and is not initially modeled or simulated for predictive purposes among the N=3 most-likely candidate behaviors. However, as the PUM performs additional actions, such as “moving away from the door to the garden,” the system may reevaluation which candidate behaviors are among the N most-likely, and begin modeling the candidate behavior of “doing laundry” when another candidate behavior (e.g., “going to the garden”) drops from the N most-likely candidate behaviors.

At block 740, the system calculates a baseline risk in performing the candidate behavior. In various embodiments, the baseline risk may be represented as a scalar or vector in the AI/ML model for an average stakeholder in performing the action. For example, changing a lightbulb in a ceiling fixture may be evaluated as a mildly risky behavior across the set of all stakeholders (e.g., due to potentially needing to use a stepladder, the potential of electrical shock, etc.), but may be riskier or less risky for certain stakeholders (e.g., shorter versus taller stakeholders who require or do not require the use of a stepladder).

In various embodiments, the risk metric (baseline or otherwise) identifies a likelihood of the candidate behavior including a safety-affecting action selected from the group consisting of: the PUM tripping or falling; the PUM experiencing a dementia episode; the PUM leaving an appliance in an active configuration after conclusion of an associated action (e.g., leaving the stove or oven on or unattended, leaving a faucet running, leaving a hair dryer or iron on, leaving a battery operated device active, etc.); the PUM dropping an object; the PUM disclosing private information to an unauthorized party; the PUM experiencing a food or medication interaction or difficulty; the PUM experiencing a bathing difficulty; and the PUM leaving a door or window open or unlocked after conclusion of an associated behavior (e.g., entering or exiting a room or the SEE).

At block 750, the system identifies a personalization factor representing the particular situation in which the behavior is being performed. For example, a time of day at which the behavior is being performed, a location at which the behavior is being performed, and a mental attitude of the PUM for being able to safely perform the candidate behavior can, separately or in combination, be used in a personalization factor.

In various embodiments, identifying the personalization factor includes prompting, for example via an audio or text-based message, the PUM or a stakeholder for care of the PUM for an assessment of the mental attitude of the PUM for being able to safely perform the candidate behavior; and receiving, in response to prompting the PUM or the stakeholder, an instruction, either verbal or textual, from the PUM or the stakeholder to set the personalization factor to a particular value associated with an indicated mental attitude. For example, a speaker in the SEE may ask the PUM, “How safely you think you can safely take a shower on your own?”, to which the PUM may respond, as detected by a microphone in the SEE, “Very safely”, “I can do that fine,” “I may need help stepping in and out”, etc., which are associated by the AI/ML system with different personalization factor values. For example, a stakeholder of a physician, spouse, responsible adult child, etc. may be prompted via an text-based message by the SEE to assess how safely a PUM is expected to be able to bathe/shower themselves, and may be presented a scale of 1-10, which corresponds to the values that the system uses in a personalization factor for the behavior.

In various embodiments, identifying the personalization factor includes identifying a hesitation or an interruption in performing the candidate action of at least a threshold amount of time after the in-progress action is performed; and increasing the risk metric (per block 760) from the baseline value to the personalized value. For example, when a PUM is engaged in a behavior of bathing/showering, and takes more than X seconds to leave the shower after action of turning off the water is complete, the hesitation of at least X seconds may be indicative that water is on the floor, the PUM is having difficulty raising a foot above a rim of the bathtub, etc., or has otherwise identified an increased risk to the situation or is experiencing difficulty in safely completing the behavior.

In various embodiments, identifying the personalization factor includes identifying initiation of performance of the candidate action after the in-progress action is performed before a duration threshold for performing the in-progress action is satisfied; and decreasing the risk metric (per block 760) from the baseline value to the personalized value. For example, a PUM may more quickly or competently perform tasks than the system expects, such as when the system has misidentified a risk factor in the environment or a limitation of the PUM, and the system may react by treating the behavior as less risky to the health, wellbeing, and safety of the PUM than initially indicated.

In various embodiments, identifying the personalization factor includes determining whether the stakeholder has paid attention (or is ignoring) an alert to a potentially risky situation to the health, wellbeing or safety of the PUM that the system has communicated to the stakeholder (e.g., per block 790). In various embodiments, determining whether a stakeholder has given attention to the alert includes identifying at least one demonstration of ignoring the alert identifiable from the state information after the alert is provided. These demonstrations may include identification of: the PUM maintaining a speed of performing the in-progress action or the candidate behavior; the PUM maintaining a field of view or gaze in the SEE that does not include a device on which the alert is provided; the PUM failing to provide a verbal or device-based acknowledgement of the alert within an acknowledgement window; and the PUM providing a verbal or device-based dismissal of the alert. In response to determining that the PUM or other stakeholder is ignoring or has not been made aware of the alert, the system may (per block 760) adjust the baseline risk upward, to indicate that the stakeholder is proceeding recklessly, that another stakeholder should be alerted, that a different form of alert should be tried, or another responsive action should be taken within the SEE.

In various embodiments, the time at which the behavior is performed or a location where the behavior is performed is used as personalization factors to identify when the PUM or a stakeholder is engaged in a behavior in an unusual time or location, which may indicate additional risks to the health, wellness or safety of the PUM.

At block 760, the system adjusts the risk metric from the baseline value to a personalized value based on the personalization factor. In various embodiments, the safety attitude value is applied as a multiplier to the baseline value, which may increase, decrease, or maintain the baseline value based. More than one personalization factor may be applied to the baseline risk assessment to yield the personalized risk assessment, with two or more of the personalization factors combining constructively or destructively (e.g., fully or partially canceling out the effect of each other).

At block 770, the system evaluates the baseline value, the personalized value, and a difference between the baseline value and the personalized value to determine whether an actionable state (versus a quiescent state) is present in the SEE. This actionable state may be in addition to or alternatively to whether the behavior is actionable, an intended or predicted outcome of the behavior is actionable, or a difference between the intended and predicted outcomes is actionable (per method 600 discussed in relation to FIG. 6). When one or more of a baseline value, the personalized value, and a difference between the baseline value and the personalized value are represent an actionable risk level for health, wellness or safety of the PUM, method 700 proceeds to one or more of block 780 and block 790; otherwise, method 700 may return to block 710 to continue monitoring the PUM.

In various embodiments, the baseline value, the personalized value, and a difference between the baseline value and the personalized value be assessed against various thresholds, Boolean checks, consensus models (e.g., among a plurality of digital twins), and combinations thereof to determine when the state in the SEE is actionable versus quiescent. For example, the system can identify a time window historically associated with the PUM performing the candidate behavior so that adjusting the risk metric from the baseline value to the personalized value increases the risk metric in response to the candidate behavior being performed outside of the time window. For example, the system can identify a location range where the stakeholder has historically performed the candidate behavior so that adjusting the risk metric from the baseline value to the personalized value increases the risk metric in response to the candidate behavior being performed outside of the location range. When the time or location is outside of the historical windows, the system may identify to a stakeholder other than the PUM that the PUM is behaving atypically, regardless of whether the action itself is risky, as this anomalous behavior, while potentially not a risk to the health, wellness or safety of the PUM may be evidence of a change in routing, a dementia event, or may affect future behaviors of the PUM.

At block 780, the system recalibrates at least one sensor of the plurality of sensors. The recalibration may be in response to an actionable state being identified from one or more of the baseline value, the personalized value, or a difference between the baseline and personalized values. Such a state identification may be a false positive (e.g., when the sensors detect a state that does not exist or offers a lower actual risk than the system has identified), require further information to act upon, or, and on or more of the sensors are therefore recalibrated within the SEE.

For example, a recalibration may be performed when the system identifies a task as having a baseline level of risk for a generalized PUM above a risk threshold even if the personalized value is below the risk threshold. Accordingly, the system can recalibrate one or more sensors to determine whether the state of the environment and behaviors being performed therein are actually actionable (e.g., to determine that the system reported a false positive for a behavior being risky) or when the PUM has a false sense of security in the riskiness of a particular behavior.

For example, a recalibration may be performed when the system identifies a task as having a baseline level risk for a generalized PUM below a risk threshold but that the personalized value is above the risk threshold. Accordingly, the system can recalibrate one or more sensors to identify environmental conditions identified (accurately or otherwise) by the PUM that the system may have not observed, or to treat the specific PUM differently from a generalized PUM. For example, a specific PUM may have a phobia, belief (supported or otherwise) that a particular task is personally risky to perform, or may be suffering an observed medical condition that renders an otherwise safe task risky to perform (e.g., when the PUM is confused, disoriented, or affected by an ongoing dementia event).

For example, a recalibration may be performed when the system identifies a difference in the baseline and personal levels of risk exceeds an anomaly threshold, even when neither the baseline nor personal levels of risk exceed the respective risk thresholds. Accordingly, the system can recalibrate one or more sensors to identify environmental conditions that are interpreted differently by the PUM than the system to identify and report on anomalous understandings of the SEE and the riskiness of various behaviors being performed therein.

In some embodiments, a recalibration may be performed when the system identifies both that 1) a difference in the baseline and personal levels of risk exceeds an anomaly threshold, and 2) at least one of the baseline value and the personalized value exceeds a risk threshold. Accordingly, the system can recalibrate one or more sensors to identify environmental conditions that are interpreted differently by the PUM than the system to identify and report on anomalous understandings of the SEE and the riskiness of various behaviors being performed therein when one of the PUM or the system views a behavior as potentially risky enough to warrant alerting a stakeholder of the PUM performing the behavior.

In various embodiments, reconfiguring a sensor can include sending a configuration command to one or more of a plurality of sensors to change a configuration of one or more sensors by: activating at least one of the plurality of sensors that is currently inactive; deactivating at least one of the plurality of sensors that is currently active; increasing a granularity of data collected by at least one of the plurality of sensors that is currently active (e.g., to report more data in a particular time period, report cached data); decreasing the granularity of data collected by at least one of the plurality of sensors that is currently active (e.g., to report less data in a particular time period); increasing a reporting rate of at least one of the plurality of sensors that is currently active; decreasing the reporting rate of at least one of the plurality of sensors; changing an optical focus of at least one of the plurality of sensors that is currently active (e.g., activate a motor to move where a camera is pointed in the SEE, activate a hardware zoom-in/zoom-out function).

Method 700 may proceed from block 780 to one or more of block 790 and block 710.

At block 790, the system sends a message to a stakeholder for care of the PUM. In various embodiments, the message may be sent to one or more devices within the SEE to alert the PUM or another stakeholder in the SEE to a behavior identified as actionable. In various embodiments, the message may be sent to one or more stakeholders outside of the SEE when a behavior is identified as actionable. In various embodiments, the stakeholders can include the PUM, a caregiver of the PUM, a friend of the PUM, a neighbor of the PUM, a family member of the PUM, an insurance provider for the PUM, a medical professional, and an emergency responder.

In various embodiments, the message may be an alert transmitted to a device within the SEE or outside of the SEE to identify that the PUM is at risk due to the behavior being performed.

Various stakeholders may be provided various messages according to different criteria so that the messages are sent in a cascade to different parties so that the system may provide a first message to a first stakeholder for care of the PUM that identifies the candidate behavior and when the first message is ignored, not responded to within a time window, or the risk evaluation increases, the system may provide a second message to a second stakeholder for care of the PUM that also identifies the candidate behavior. In various embodiments, the message may be provided as part of a token, where each of the first and second stakeholders are provided the same tokenized message, where the first stakeholder is provided a first decryption key for accessing the message, and is denied access to the second message; and the second stakeholder is provided a second decryption key for accessing the second message, and is denied access to the first message so that both stakeholders have access to the relevant sections of the tokenized message and are not given access to the other sections. Accordingly, the token may be provisioned to the first stakeholder and the second stakeholder at the same time, but the system may provide the decryption keys at different times, thereby allowing the system to manage bandwidth for communication more effectively, and permit the sharing or forwarding of the message among stakeholders while retaining control over access to the contents of the tokenized message. For example, the system may provide the single tokenized message to both the first and second stakeholders in response to the baseline value or the personalized value exceeding the elevated risk threshold or the difference exceeding the anomaly threshold and the triggering condition for the second stakeholder to be provided access to the included message, where the system transmits the second decryption key for accessing the included message to the second stakeholder in response to triggering condition being satisfied.

In various embodiments, the message may include a command to a system or device within the SEE to display or output a message to persons present within the SEE that identify the risks and conditions that the system used to identify the risk. Additionally or alternative, the command may cause one or more systems in the SEE to take mitigating actions, such as, for example, shutting off an appliance; engaging or disengaging a lock on a door or window; opening or closing a door or window; turning on or off a light; and terminating a telephone call.

Method 700 may proceed from block 790 to one or more of block 780 and block 710.

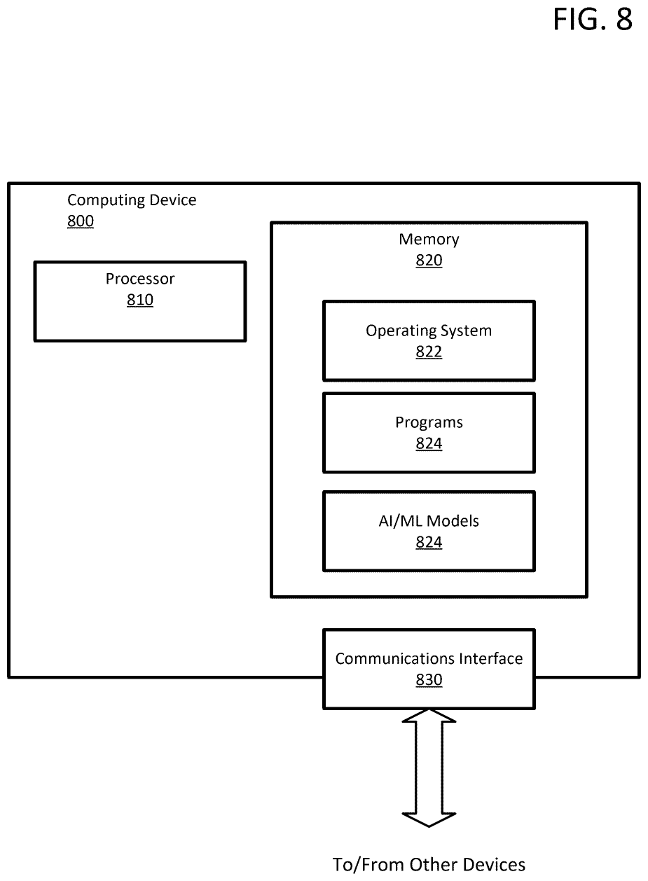

FIG. 8 illustrates an example computing device 800, as may be used as a controller in a SEE to monitor a PUM, as part of a sensor monitoring a PUM, as part of a central or distributed service providing calibration systems for generating and curating AI/ML models for distribution to the SEEs, and the like, according to embodiments of the present disclosure. For example, the computing device 800 may perform the operations set out in method 600 or method 700. The computing device 800 may include at least one processor 810, a memory 820, and a communication interface 830.

The processor 810 may be any processing unit capable of performing the operations and procedures described in the present disclosure (e.g., method 600). In various embodiments, the processor 810 can represent a single processor, multiple processors, a processor with multiple cores, and combinations thereof.

The memory 820 is an apparatus that may be either volatile or non-volatile memory and may include RAM, flash, cache, disk drives, and other computer readable memory storage devices. Although shown as a single entity, the memory 820 may be divided into different memory storage elements such as RAM and one or more hard disk drives. As used herein, the memory 820 is an example of a device that includes computer-readable storage media, and is not to be interpreted as transmission media or signals per se.

As shown, the memory 820 includes various instructions that are executable by the processor 810 to provide an operating system 822 to manage various features of the computing device 800 and one or more programs 824 to provide various functionalities to users of the computing device 800, which include one or more of the features and functionalities described in the present disclosure (e.g., method 600, method 700). One of ordinary skill in the relevant art will recognize that different approaches can be taken in selecting or designing a program 824 to perform the operations described herein, including choice of programming language, the operating system 822 used by the computing device 800, and the architecture of the processor 810 and memory 820. Accordingly, the person of ordinary skill in the relevant art will be able to select or design an appropriate program 824 based on the details provided in the present disclosure.

Additionally, the memory 820 may include one or more AI/ML models 826 that interact with, are trained by, or are curated by the programs 824. The AI/ML models 826 may include AI/ML models that are available for use to identify various behaviors that place the health, wellness or safety of the PUM at risk, and take appropriate action to avoid to mitigate that risk, as described herein.

The communication interface 830 facilitates communications between the computing device 800 and other devices, including sensors in a SEE, which may also be computing devices as described in relation to FIG. 8. In various embodiments, the communication interface 830 includes antennas for wireless communications and various wired communication ports. The computing device 800 may also include or be in communication, via the communication interface 830, one or more input devices (e.g., a keyboard, mouse, pen, touch input device, etc.) and one or more output devices (e.g., a display, speakers, a printer, etc.).

Although not explicitly shown in FIG. 8, it should be recognized that the computing device 800 may be connected to one or more public or private networks via appropriate network connections via the communication interface 830. It will also be recognized that software instructions may also be loaded into a non-transitory computer readable medium, such as the memory 820, from an appropriate storage medium or via wired or wireless means.

Systems, methods, and apparatuses of the present disclosure may be implemented on a variety of devices, such as but not limited to IPUs, DPUs, CPUs, GPUs, ASICS, FPGAs, DSPs, or any other device capable of processing data. Instructions for performing the same may be provided as hardware or firmware on any computer-readable medium including volatile and non-volatile forms of memory. Particular implementations of techniques of the present disclosure may be structured in any number of ways, including but not limited to a modular program architecture, a monolithic program architecture, on a single device, and distributed across more than one device or processor.

Although certain figures and descriptions have been provided, many additional variations and modifications will be apparent to those of skill in the art. It will be appreciated that presenting all possible variations and modifications is an impractical task, and thus any sequence, particular structural or device implementation, or underlying technique of the present disclosure may be substituted or modified to meet the needs of particular implementations, and that doing so may not depart from the scope of the present disclosure. It will therefore be appreciated that the examples presented herein are presented for illustrative purposes only, and are in no way intended to be limiting of a scope of the present disclosure. It will also be apparent to any individual of skill in the art that various embodiments described herein and elements thereof may be combined as needed to suit any particular implementation, and that doing so does not depart from the scope of the present disclosure. As such, the scope of the present disclosure is not to be understood as being limited by the figures or specification presented herein; the scope of the present disclosure should instead be understood in a context of the appended claims and their equivalents.

Certain terms are used throughout the description and claims to refer to particular features or components. As one skilled in the art will appreciate, different persons may refer to the same feature or component by different names. This document does not intend to distinguish between components or features that differ in name but not function.

As used herein, the term “optimize” and variations thereof, is used in a sense understood by data scientists to refer to actions taken for continual improvement of a system relative to a goal. An optimized value will be understood to represent “near-best” value for a particular reward framework, which may oscillate around a local maximum or a global maximum for a “best” value or set of values, which may change as the goal changes or as input conditions change. Accordingly, an optimal solution for a first goal at a particular time may be suboptimal for a second goal at that time or suboptimal for the first goal at a later time. Similarly, terms of “minimize” and “maximize” shall generally be understood to refer to optimizing for a “best” lowest value or highest value, respectively, and may include the identification of local minima, local maxima, a global minimum, or a global maximum, which can vary at different times or under new goals or conditions.

As used herein, “about,” “approximately” and “substantially” are understood to refer to numbers in a range of the referenced number, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole numbers, or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in the present disclosure, the term “or” is to be interpreted in the inclusive sense and not the exclusive sense unless explicitly stated otherwise or when clear from the context. Accordingly, recitation of “A or B” is intended to cover the sets of A, B, and A-B, where the sets may include one or multiple instances of a particular member (e.g., A-A, A-A-A, A-A-B, etc.) and any ordering thereof.

As used in the present disclosure, a phrase referring to “at least one of” a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, A-C, and A-B-C, where the sets may include one or multiple instances of a particular member (e.g., A-A, A-A-A, A-A-B, A-A-B-B-C-C-C, etc.) and any ordering thereof. For avoidance of doubt, the phrase “at least one of A, B, and C” shall not be interpreted to mean “at least one of A, at least one of B, and at least one of C”.

As used in the present disclosure, the term “including” is intended to encompass “including but not limited to” and unless expressly noted otherwise, leaves open the possibility of additional options or features.

As used in the present disclosure, the term “determining” encompasses a variety of actions that may include calculating, computing, processing, deriving, investigating, identifying, looking up (e.g., via a table, database, or other data structure), ascertaining, receiving (e.g., receiving information), accessing (e.g., accessing data in a memory), retrieving, resolving, selecting, choosing, establishing, and the like.

Without further elaboration, it is believed that one skilled in the art can use the preceding description to use the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

Within the claims, reference to an element in the singular is not intended to mean “one and only one” unless specifically stated as such, but rather as “one or more” or “at least one”. Unless specifically stated otherwise, the term “some” refers to one or more. No claim element is to be construed under the provision of 35 U.S.C. § 112(f) unless the element is expressly recited using the phrase “means for” or “step for”. All structural and functional equivalents to the elements of the various embodiments described in the present disclosure that are known or come later to be known to those of ordinary skill in the relevant art are expressly incorporated herein by reference and are intended to be encompassed by the claims. Moreover, nothing disclosed in the present disclosure is intended to be dedicated to the public regardless of whether such disclosure is explicitly recited in the claims.

We claim:

1. A method, comprising:
measuring, via a plurality of sensors disposed in a Sensor Enabled Environment (SEE), state information of the SEE and a person under monitoring (PUM) within the SEE;
identifying, using the state information, an in-progress behavior affecting the PUM in the SEE;
identifying an intended outcome of the in-progress behavior;
predicting, using the state information and the in-progress behavior, a predicted outcome of the in-progress behavior; and
in response to identifying that a difference between the intended outcome and the predicted outcome represents an actionable risk level for health, wellness or safety of the PUM:
identifying a responsive action to the in-progress behavior to reduce the actionable risk level to the PUM; and
performing the responsive action.

2. The method of claim 1, wherein the in-progress behavior is performed at least in part by a stakeholder for care of the PUM other than the PUM, wherein the other stakeholder is selected from the group consisting of:
a caregiver of the PUM;
a friend of the PUM;
a neighbor of the PUM;
a family member of the PUM;
an insurance provider for the PUM;
a medical professional; and
an emergency responder.

3. The method of claim 1, wherein the in-progress behavior is performed, at least in part, by the PUM.

4. The method of claim 1, wherein performing the responsive action includes:
transmitting a message to the PUM or at least one other stakeholder for care of the PUM that identifies the predicted outcome of the in-progress behavior.

5. The method of claim 1, wherein performing the responsive action includes:
transmitting a message to the PUM or at least one other stakeholder for care of the PUM that identifies an alternative behavior associated with the intended outcome with a lower risk assessment than the in-progress behavior.

6. The method of claim 1, wherein performing the responsive action includes:
reconfiguring at least one sensor of the plurality of sensors by sending a configuration command for the at least one sensor of the plurality of sensors from the group consisting of:
activating the at least one sensor of the plurality of sensors;
deactivating the at least one sensor of the plurality of sensors; and
increasing a granularity of data collected by the at least one sensor of the plurality of sensors;
decreasing the granularity of data collected by the at least one sensor of the plurality of sensors;
increasing a reporting rate of the at least one sensor of the plurality of sensors;
decreasing the reporting rate of the at least one sensor of the plurality of sensors; and
changing an optical focus of the at least one sensor of the plurality of sensors.

7. The method of claim 1, wherein performing the responsive action includes:
transmitting a control signal to a device disposed in the SEE that performs a safety action selected from the group consisting of:
shutting off an appliance;
engaging or disengaging a lock on a door or window;
opening or closing a door or window;
turning on or off a light; and
terminating a telephone call.

8. The method of claim 1, wherein identifying whether the difference between the intended outcome and the predicted outcome represents the actionable risk level for health, wellness or safety of the PUM is determined, at least in part, by analyzing at least one of:
data from a health care profile (HCP) of the PUM;
historical behavior patterns of the PUM; and
contextual data of the SEE for a time or location where the in-progress behavior is being performed.

9. The method of claim 1, wherein the in-progress behavior includes movement within the SEE and the actionable risk level for health, wellness or safety of the PUM is identified based on a status selected from the group consisting of:
a state of an appliance in the SEE;
a light level in the SEE;
a time of day;
a temperature in the SEE;
a presence of a person or animal in the SEE;
a medication taken by the PUM;
a food or beverage consumed by the PUM; and
a presence of liquid on a floor of the SEE.

10. The method of claim 1, wherein the in-progress behavior includes a conversation that includes the PUM and the actionable risk level for health, wellness or safety of the PUM is identified based on sharing private information with another party to the conversation or present in the SEE that is not authorized to receive the private information.

11. The method of claim 1, wherein the in-progress behavior includes a repeated action not necessary to be performed more than once for achieving the intended outcome, and the actionable risk level for health, wellness or safety of the PUM is identified with a dementia event.

12. The method of claim 1, wherein the actionable risk level for health, wellness or safety of the PUM is identified with a mental state event associated with an emotional response by the PUM, wherein the emotional response includes the PUM uttering at least one word or phrase, wherein the emotional response is selected from the group consisting of:
yelling;
crying;
screaming;
hyperventilating;
tachycardia;
a spike or a drop in blood pressure of the PUM;
a self-soothing behavior; and
a pain response.

13. The method of claim 1, wherein the actionable risk level for health, wellness or safety of the PUM is identified is identified according to a baseline level associated with the in-progress behavior modified by at least one of:
a time-based adjustment for when the in-progress behavior is being performed;
a location-based adjustment for where the in-progress behavior is being performed; and
a safety attitude-based adjustment for how confident the PUM or another stakeholder is in performing actions of the in-progress behavior.

14. The method of claim 13, wherein identifying the safety attitude-based adjustment includes:
prompting the PUM or a stakeholder for care of the PUM for an assessment of a mental or physical ability of the PUM for being able to safely perform the in-progress behavior; and
receiving, in response to prompting the PUM or the stakeholder, an instruction from the PUM or the stakeholder to set the safety attitude-based adjustment to a particular value.

15. The method of claim 13, wherein identifying the safety attitude-based adjustment includes:
identifying a hesitation or an interruption in performing actions comprising the in-progress behavior of at least a threshold amount of time; and
increasing a risk metric for the in-progress behavior from a baseline value to a personalized value for the PUM.

16. The method of claim 13, further comprising:
in response to a baseline value for a risk metric for the in-progress behavior exceeding a safety threshold, conveying an alert to the SEE that the safety threshold has been exceeded;
identifying whether the PUM has given attention to the alert; and
wherein identifying the safety attitude-based adjustment includes:
increasing the risk metric from the baseline value to a personalized value for the PUM in response to identifying that the PUM has not given attention to the alert,
wherein identifying that the PUM has not given attention to the alert includes at least one demonstration of ignoring the alert identifiable from the state information after the alert is provided, the at least one demonstration selected from the group consisting of:
the PUM maintaining a speed of performing actions of the in-progress behavior;
the PUM maintaining a field of view or gaze in the SEE that does not include a device on which the alert is provided;
the PUM failing to provide a verbal or device-based acknowledgement of the alert within an acknowledgement window; and
the PUM providing a verbal or device-based dismissal of the alert.

17. The method of claim 1, wherein the plurality of sensors includes a personal sensor that is worn by, carried by, or implanted in the PUM.

18. The method of claim 1, wherein the plurality of sensors includes an environmental sensor that is disposed in the SEE and not worn by, carried by, or implanted in the PUM.

19. The method of claim 1, further comprising:
using a Large Language Model or Large Context Model artificial intelligence or machine learning (LLM/LCM AI/ML) system to identify the in-progress behavior, the intended outcome, or the predicted outcome.

20. A system comprising:
a processor; and
a memory, including instructions that, when executed by the processor, perform operations comprising:
measuring, via a plurality of sensors disposed in a Sensor Enabled Environment (SEE), state information of the SEE and a person under monitoring (PUM) within the SEE;
identifying, using the state information, an in-progress behavior affecting the PUM in the SEE;
identifying an intended outcome of the in-progress behavior;
predicting, using the state information and the in-progress behavior, a predicted outcome of the in-progress behavior; and
in response to identifying that a difference between the intended outcome and the predicted outcome represents an actionable risk level for health, wellness or safety of the PUM:
identifying a responsive action to the in-progress behavior to reduce the actionable risk level to the PUM; and
performing the responsive action.
